(19) United States
(12) Patent Application Publication
(10) Pub. No.: US 2024/0268935 A1
(43) Pub. Date: Aug. 15, 2024

(54) INTRAORAL SCANNING

(71) Applicant: Dentlytec G.P.L. LTD., Hod Hasharon (IL)

(72) Inventors: Benny PESACH, Rosh Haayin (IL); Amitai REUVENNY, Kfar-Saba (IL); Ygael GRAD, Tel-Aviv (IL); Blanc Zach LEHR, Tel-Aviv (IL)

(73) Assignee: Dentlytec G.P.L. LTD., Hod Hasharon (IL)

(21) Appl. No.: 18/681,028

(22) PCT Filed: Aug. 2, 2022
(86) PCT No.: PCT/IL2022/050833

§ 371 Date: Feb. 2, 2024

Related U.S. Application Data
(60) Provisional application No. 63/278,075, filed on Nov. 10, 2021.
(60) Provisional application No. 63/229,040, filed on Aug. 3, 2021.

(51) Int. Cl. A61C 9/00; G06T 1/00; G06T 7/00; G06T 17/00

(52) U.S. Cl. CPC A61C 9/0053; G06T 1/0007; G06T 7/00; G06T 17/00; G06T 2207/30036

(57) ABSTRACT

A dental add-on for an electronic communication device including an imager, the dental add-on comprising: a body comprising a distal portion sized and shaped to be at least partially inserted into a human mouth, the distal portion comprising a slider configured to mechanically guide movement of the add-on along a dental arch; and an optical path extending from the imager of the electronic communication device, through the body to the slider, and configured to adapt a FOV of the imager for dental imaging.

### RELATED APPLICATIONS

This application claims the benefit of priority of U.S. Provisional Patent Application No. 63/229,040 filed on 3 Aug. 2021, and from U.S. Provisional Patent Application No. 63/278,075 filed on 10 Nov. 2021, the contents of which are incorporated herein by reference in their entirety.

### FIELD AND BACKGROUND OF THE INVENTION

Embodiments of the present disclosure relate to dental measurement devices and methods and, more particularly, but not exclusively, to intraoral scanning devices and methods.

### SUMMARY OF THE INVENTION

Following is a non-exclusive list including some examples of embodiments of inventions disclosed herein. The disclosure also includes embodiments which include fewer than all the features in an example and embodiments using features from multiple examples, also if not expressly listed below.

Example 1. A dental add-on for an electronic communication device including an imager, said dental add-on comprising:
a body comprising a distal portion sized and shaped to be at least partially inserted into a human mouth, said distal portion comprising a slider configured to mechanically guide movement of the add-on along a dental arch; and
an optical path extending from said imager of said electronic communication device, through said body to said slider, and configured to adapt a FOV of said imager for dental imaging.

Example 2. The dental add-on according to example 1, wherein said optical path emanates from said slider towards one or more dental feature, when said distal portion is positioned within a mouth.

Example 3. The dental add-on according to any one of examples 1-2, wherein said optical path is provided by one or more optical element guiding light within said optical path.

Example 4. The dental add-on according to example 3, wherein said optical path comprises at least one optical element for splitting the light path into more than one direction.

Example 5. The dental add-on according to example 4, wherein said light path emerges in one or more direction from said slider.

Example 6. The dental add-on according to example 5, wherein said optical element for splitting said light path is located at said slider.

Example 7. The dental add-on according to any one of examples 1-6, wherein said slider comprises:
a first mirror configured to direct light between the add-on and a first side of a dental feature; and
a second mirror configured to direct light between the add-on and a second side of said dental feature.

Example 8. The dental add-on according to example 7, wherein a first portion of light transferred along said add-on to said distal end is directed by said first mirror to said first side of said dental feature, and a second portion of said light transferred is directed by said second mirror to said second side of said dental feature.

Example 9. The dental add-on according to any one of examples 1-8, wherein said slider includes at least one wall, extending towards teeth surfaces during scanning which is positioned adjacent a tooth surface during scanning to guide scan movements.

Example 10. The dental add-on according to any one of examples 1-9, wherein said slider includes at least two walls, meeting at an angle to each other of 45-125° where, during scanning with the add-on, a first wall is positioned adjacent a first tooth surface and a second wall is positioned adjacent a second tooth surface during scanning to guide scan movements.

Example 11. The dental add-on according to any one of examples 1-10, wherein said slider includes a cavity sized and shaped to hold at least a portion of a dental feature aligned to said optical path so that at least a portion light emitted by said dental feature enters said optical path to arrive for sensing at said imager.

Example 12. The dental add-on according to any one of examples 1-11, wherein an orientation of said slider, with respect to said distal portion is adjustable.

Example 13. The dental add-on according to any one of examples 1-12, wherein said add-on includes a pattern projector aligned with said optical path to illuminate dental features adjacent to said slider with patterned light.

Example 14. The dental add-on according to example 13, wherein said pattern projector projects a pattern which, after passing through said optical path illuminates dental features with a pattern which is aligned to one or more wall of said slider.

Example 15. The dental add-on according to example 14, wherein said pattern projector projects parallel lines, where the parallel lines, when incident on dental features, are aligned with a perpendicular component to a plane of one or more guiding wall of said slider.

Example 16. A dental add-on for an electronic communication device including an imager, said dental add-on comprising:
a body comprising an elongate distal portion sized and shaped to be at least partially inserted into a human mouth, said distal portion comprising a slider having at least one wall directed towards dental features and configured to mechanically guide movement of the add-on along a dental arch, where said at least one slider wall has an adjustable orientation with respect to a direction of elongation of said distal portion; and
an optical path extending from said imager of said electronic communication device, through said body to said slider, and configured to adapt a FOV of said optical element for dental imaging.

Example 17. The dental add-on according to example 16, wherein said slider includes one or more optical element for splitting said optical path, and where these optical elements have adjustable orientation along with said at least one slider wall.

Example 18. The dental add-on according to any one of examples 16-17, wherein said at least one slider wall configured to adjust orientation under force applied to said at least one slider wall by dental features during movement of the slider along dental features of a jaw.

Example 19. The dental add-on according to any one of examples 16-18, wherein said slider is coupled to said distal portion by a joint, where said slider is rotatable with respect to said joint, in an axis which has a perpendicular component with respect to a direction of elongation of said distal portion.

Example 20. The dental add-on according to any one of examples 1-19, comprising a probe extending from said add-on distal portion towards dental features.

Example 21. The dental add-on according to example 20, wherein said probe is sized and shaped to be inserted between a tooth and gum tissue.

Example 22. A method of dental scanning comprising:
coupling an add-on to a portable electronic device including an imager, said coupling aligning an optical path of said add-on to a FOV of said imager, where said optical path emanates from a slider disposed on a distal portion of said add-on configured to be placed within a human mouth; and
moving said slider along a jaw, while adjusting an angle of said slider with respect to said distal portion.

Example 23. The method of example 22, wherein said adjusting is by said moving.

Example 24. A method of dental scanning:
coupling an add-on to a portable electronic device including an imager, said coupling aligning an optical path of said add-on to a FOV of said imager, where said optical path emanates from a distal portion of said add-on which is sized and shaped to be placed within a human mouth;
acquiring, using said imager:
a plurality of narrow range images of one or more dental feature;
at least one wide range image of said one or more dental feature, where said wide range image is acquired from a larger distance from said dental feature than said plurality of narrow range images; and
generating a model of said dental features from said plurality of close range images and said at least one wide range image.

Example 25. The method of dental scanning according to example 24, wherein said plurality of narrow range images and said at least one wide range image are acquired through said add-on.

Example 26. The method of dental scanning according to example 24, wherein said acquiring comprises:
acquiring a plurality of narrow range images through said add-on; and
acquiring at least one wide range image by said portable electronic device.

Example 27. The method according to example 24, wherein said at least one wide range image is acquired through said add-on using an imager FOV which emanates from said add-on distal portion with larger extent than an imager FOV used to acquire said narrow range images.

Example 28. The method according to example 24, wherein said at least one wide range image is acquired using an imager of said electronic device not coupled to said add-on.

Example 29. The method according to any one of examples 24-28, wherein said portable electronic device is an electronic communication device having a screen.

Example 30. The method according to any one of examples 24-29, wherein said model is a 3D model.

Example 31. The method according to any one of examples 24-30, wherein said generating comprises generating a model using said narrow range images and correcting said model using said at least one wide range image.

Example 32. The method according to any one of examples 24-31, wherein said plurality of images are acquired of dental features illuminated with patterned light.

Example 33. The method according to any one of examples 24-31, wherein said add-on optical path transfers patterned light produced by a pattern projector to dental surfaces.

Example 34. The method according to any one of examples 24-33, wherein said at least one wide range image includes dental features not illuminated by patterned light.

Example 35. A method of dental scanning comprising:
coupling an add-on to a portable electronic device including an imager, said coupling aligning an optical path of said add-on to a FOV of said imager, where said optical path emanates from a distal portion of said add-on which is sized and shaped to be placed within a human mouth;
controlling image data acquired said imager by performing one or more of:
   disabling one or more automatic control feature of said electronic device imager; and
   determining image processing compensation for said one or more automatic control feature;
   acquiring, using said imager, a plurality of images of one or more dental feature; and
   if imaging processing compensation has been determined, processing said plurality of images according to said processing compensation.

Example 36. The method according to example 35, wherein said automatic control feature is OIS control.

Example 37. The method according to example 36, wherein said determining is by using sensor data used by a processor of said electronic device to determine said OIS control.

Example 38. The method according to example 36, wherein said disabling is by one or more of:
a magnet of said add-on positioned adjacent to said imager; and
software disabling of said OIS control, by software installed on said electronic device.

Example 39. A method of dental scanning comprising:
illuminating one or more dental feature with polarized light;
polarizing returning light from said one or more dental feature;
acquiring one or more image of said returning light; and
generating a model of said one or more dental feature, using said one or more image.

Example 40. The method according to example 39, wherein said illuminating and said acquiring a through an optical path of an add-on coupled to a portable electronic device.

Example 41. A dental add-on for an electronic communication device including an imager comprising:
a body comprising a distal portion sized and shaped to be at least partially inserted into a human mouth;
an optical path extending from said imager of said electronic communication device, and configured to adapt a FOV of said imager for dental imaging, said optical path including a polarizer; and
a polarized light source emanating light from said distal portion, said polarized light source comprising one or more of:
   a polarizer aligned with an illuminator of said imager or an illuminator of said add-on; and
   a polarized light source of said add-on.

Example 42. The dental add-on according to example 41, wherein said distal portion comprises a slider configured to mechanically guide movement of the add-on along a dental arch and where said optical path passes through said body to said slider.

Example 43. A kit comprising:
an add-on according to any one of examples 1-23 or any one of examples 41-42;
a calibration element comprising:
one or more calibration marking; and
a body configured to position one or more of:
   an FOV of imager of an electronic device so that the FOV includes at least a portion of said one or more calibration marking; and
   said add-on so that said optical path of said add-on extends to include at least apportion of said one or more calibration marking.

Example 44. A dental add-on for an electronic communication device including an imager comprising:
a body comprising a distal portion sized and shaped to be at least partially inserted into a human mouth; and
an optical path extending from said imager of said electronic communication device, and configured to adapt a FOV of said imager for dental imaging, said optical path includes a single element which provides both optical power and light patterning.

Example 45. The dental add-on according to any one of examples 1-16, wherein said optical path includes a single element which provides both optical power and light patterning

Example 46. A method of dental scanning comprising:
acquiring a plurality of images of dental features illuminated by patterned light while moving a final optical element of an imager along at least a portion of a jaw where, for one or more position along the jaw, performing one or more of:
illuminating one or more dental feature with polarized light, and polarizing returned light to an imager to acquire one or more polarized light image;
illuminating one or more dental feature with UV light and acquiring one or more image of the one or more dental feature;
illuminating dental feature/s with NIR light and acquiring one or more image of the one or more dental feature;
generating a 3D model of said dental features using said plurality of images of dental features illuminated by patterned light;
detailing said model using data determined from one or more of:
said one or more image acquired of one or more dental feature illuminated with polarized light;
said one or more image acquired of one or more dental feature illuminated with UV light; and
said one or more image acquired of one or more dental feature illuminated with NIR light.

Unless otherwise defined, all technical and/or scientific terms used herein have the same meaning as commonly understood by one of ordinary skill in the art to which the embodiments and corresponding inventions thereof pertain. Although methods and materials similar or equivalent to those described herein can be used in the practice or testing of embodiments, exemplary methods and/or materials are described below. In case of conflict, the patent specification, including definitions, will control. In addition, the materials, methods, and examples are illustrative only and are not intended to be necessarily limiting.

Implementation of the method and/or systems disclosed herein can involve performing or completing selected tasks manually, automatically, or a combination thereof. Moreover, according to actual instrumentation and equipment of embodiments of the methods and/or systems disclosed herein, several selected tasks could be implemented by hardware, by software or by firmware or by a combination thereof using an operating system.

For example, hardware for performing selected tasks according to embodiments of inventions of the disclosure could be implemented as a chip or a circuit. As software, selected tasks according to some embodiments could be implemented as a plurality of software instructions being executed by a computer using any suitable operating system. In an exemplary embodiment, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Unless otherwise defined, all technical and/or scientific terms used herein have the same meaning as commonly understood by one of ordinary skill in the art to which an invention pertains. Although methods and materials similar or equivalent to those described herein can be used in the practice or testing of embodiments herein, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments may be embodied as a system, method or computer program product. Accordingly, some embodiments may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments may take the form of a computer program product embodied in one or more computer readable medium(s) having computer readable program code embodied thereon. Implementation of the method and/or system of some embodiments can involve performing and/or completing selected tasks manually, automatically, or a combination thereof. Moreover, according to actual instrumentation and equipment of some embodiments of methods and/or systems disclosed herein, several selected tasks could be implemented by hardware, by software or by firmware and/or by a combination thereof, e.g., using an operating system.

For example, hardware for performing selected tasks according to some embodiments could be implemented as a chip or a circuit. As software, selected tasks according to some embodiments could be implemented as a plurality of software instructions being executed by a computer using any suitable operating system. In an exemplary embodiment, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments may be described below with reference to flowchart illustrations and/or block diagrams of methods, apparatus (systems) and computer program products according to some embodiments. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such inspecting objects, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE DRAWINGS

Some embodiments are herein described, by way of example only, with reference to the accompanying drawings. With specific reference now to the drawings in detail, it is stressed that the particulars shown are by way of example and for purposes of illustrative discussion of disclosed embodiments. In this regard, the description taken with the drawings makes apparent to those skilled in the art how embodiments may be practiced.

FIG. 1 is a simplified schematic of a dental measurement system, according to some embodiments;

FIG. 2A is a flowchart of a method of intraoral scanning, according to some embodiments;

FIG. 2B is a flowchart of a method, according to some embodiments;

FIG. 3A is a simplified schematic side view of an add-on connected to a smartphone, according to some embodiments;

FIG. 3B is a simplified schematic cross sectional view of an add-on, according to some embodiments;

FIG. 3C is a simplified schematic cross sectional view of an add-on, according to some embodiments;

FIG. 4 is flowchart of a method of oral measurement, according to some embodiments;

FIG. 5A is a simplified schematic of an image acquired, according to some embodiments;

FIG. 5B is a simplified schematic of an image acquired, according to some embodiments;

FIGS. 5C-E are simplified schematics of patterned illumination with respect to a dental feature, during scanning, according to some embodiments;

FIGS. 5F-H are simplified schematics of patterned illumination with respect to a dental feature, during scanning, according to some embodiments;

FIG. 6 is a simplified schematic side view of an add-on connected to a smartphone, according to some embodiments;

FIG. 7 is a simplified schematic side view of an add-on connected to a smartphone, according to some embodiments;

FIG. 8A is a simplified schematic top view of a portion of an add-on, according to some embodiments;

FIG. 8B is a simplified schematic cross sectional view of an add-on, according to some embodiments;

FIG. 8C is a simplified schematic cross sectional view of an add-on, according to some embodiments;

FIG. 8D is a simplified schematic cross sectional view of an add-on, according to some embodiments;

FIG. 8E is a simplified schematic cross sectional view of an add-on, according to some embodiments;

FIG. 9A is a simplified schematic side view of an add-on connected to a smartphone, according to some embodiments;

FIG. 9B is a simplified schematic cross sectional view of an add-on, according to some embodiments;

FIG. 10 is a simplified schematic side view of an add-on connected to a smartphone, according to some embodiments;

FIG. 11 is a simplified schematic side view of an add-on connected to an optical device, according to some embodiments;

FIG. 12 is a simplified schematic side view of an add-on connected to a smartphone, according to some embodiments;

FIG. 13A is a simplified schematic cross sectional view of a slider of an add-on, according to some embodiments;

FIG. 13B is a simplified schematic side view of a slider, according to some embodiments;

FIG. 13C is a simplified schematic cross sectional view of a slider of an add-on, according to some embodiments;

FIG. 13D is a simplified schematic side view of a slider, according to some embodiments;

FIG. 13E is a simplified schematic cross sectional view of a slider of an add-on, according to some embodiments;

FIG. 13F is a simplified schematic cross sectional view of a slider of an add-on, according to some embodiments;

FIG. 13G is a simplified schematic side view of a slider, according to some embodiments;

FIG. 14A-B are simplified schematics illustrating scanning a jaw with an add-on, according to some embodiments;

FIG. 14C is a simplified schematic top view of an add-on with respect to dental features, according to some embodiments;

FIG. 15 is a simplified schematic top view of an add-on connected to a smartphone, with respect to dental features, according to some embodiments;

FIG. 16A is a simplified schematic cross section of an add-on, according to some embodiments;

FIG. 16B is a simplified schematic of a portion of an add-on, according to some embodiments;

FIG. 17 is a simplified schematic top view of an add-on with respect to dental features, according to some embodiments;

FIG. 18A is a simplified schematic top view of an add-on connected to a smartphone, with respect to dental features, according to some embodiments;

FIG. 18B is a simplified schematic top view of an add-on connected to a smartphone, with respect to dental features, according to some embodiments;

FIG. 18C is a simplified schematic cross section view of an add-on, according to some embodiments;

FIG. 19A is a simplified schematic cross sectional view of an add-on, according to some embodiments;

FIG. 19B is a simplified schematic cross sectional view of an add-on, according to some embodiments;

FIG. 20A is a simplified schematic of an optical element, according to some embodiments;

FIG. 20B is a simplified schematic of an optical element, according to some embodiments;

FIG. 21 is a simplified schematic of a projector, according to some embodiments;

FIG. 22 is a flowchart of a method of dental monitoring, according to some embodiments;

FIG. 23 is a flowchart of a method of dental measurement, according to some embodiments;

FIG. 24A is a simplified schematic of an add-on attached to a smartphone, according to some embodiments;

FIG. 24B is a simplified schematic of an add-on, according to some embodiments;

FIG. 24C is a simplified schematic side view of an add-on, according to some embodiments;

FIG. 25 is a simplified schematic side view of an add-on connected to a smartphone, according to some embodiments;

FIG. 26A is a simplified schematic side view of an add-on connected to a smartphone, according to some embodiments;

FIG. 26B is a simplified cross sectional view of an add-on, according to some embodiments;

FIG. 26C is a simplified cross sectional view of an add-on, according to some embodiments;

FIG. 26D is a simplified cross sectional view of distal end of an add-on, having a probe, where the probe is in a retracted configuration, according to some embodiments;

FIG. 27A is a simplified schematic of an add-on within a packaging, according to some embodiments;

FIG. 27B is a simplified schematic illustrating calibration of a smartphone using a packaging, according to some embodiments;

FIG. 27C is a simplified schematic illustrating calibration of a smartphone attached to an add-on using a packaging, according to some embodiments; and

FIG. 28 is a simplified cross sectional view of an add-on, according to some embodiments.

### DESCRIPTION OF SPECIFIC EMBODIMENTS OF THE INVENTION

Embodiments of the present disclosure relate to dental measurement devices and methods and, more particularly, but not exclusively, to intraoral scanning devices and methods.

### Overview

A broad aspect of some embodiments relate to case and/or rapidity of collection of dental measurements, for example, by a subject, of the subject's mouth, herein termed “self-scanning”. In some embodiments, scanning is in a home and/or non-clinical setting. In some embodiments, self-scanning should be taken to also encompass, for example, an individual scanning the subject, e.g., an adult scanning a child.

In some embodiments, scanning is performed using a smartphone attached to an add-on. In some embodiments, scanning is performed using an electronic device including an imager (e.g., intraoral scanner IOS). Although description in this document is generally of an add-on attached to a smartphone, it should be understood that add-ons described with respect to a smartphone, in some embodiments, are configured to be attached to an electronic device including an imager e.g., an IOS. In some embodiments, the imager of the electronic device (e.g., of an IOS) is connected (e.g., wirelessly e.g., via cable/s) to a smartphone and/or to another processing unit.

Where the add-on transfers one or more optical path (“add-on” also herein termed “periscope”) from the smartphone or electronic device e.g., to a portion (e.g., distal end) of the add-on which, in some embodiments, is sized and/or shaped to be inserted into a human mouth.

In some embodiments, scanning includes swiping movement of the add-on during scanning. Where, in some embodiments swiping includes moving the add-on with respect to dental features, for example, the moving scanning at least a portion of a dental arc, the portion including more than one tooth or 2-16, or 1-8 teeth, for example, the entire arc, half an arc. Where in some embodiments a half arch is a portion of the arch extending from an end tooth (e.g., molar) to a central tooth (e.g., incisor).

In some embodiments, a swipe movement captures a single side of teeth. For example, the occlusal, the lingual, or the buccal sides. In some embodiments a swipe captures two sides of the teeth, for example occlusal and buccal or occlusal and lingual.

A broad aspect of some embodiments relates to a portion of the add-on being configured for rapid and/or easy scanning of dental features. For example, having size and/or shape and/or optical feature/s which enable rapid and/or easy scanning. In some embodiments, the portion (e.g., to which the add-on transfers one or more optical path and/or is sized and/or shaped to be inserted into a human mouth) is a slider.

An aspect of some embodiments, relates to a slider including a cavity which is sized and/or shaped to receive dental feature/s and/or to guide movement of the add-on within the mouth e.g., by forming one or more barrier to movement of the add-on in one or more direction, with respect to the dental feature/s.

For example, in some embodiments, at least a portion of dental feature/s (e.g., teeth) closely fit into the cavity. Where, for example, the volume of the cavity, when holding and/or aligned with one or more tooth is at least 50%, or at least 80%, or lower or higher or intermediate percentages, filled with the tooth or teeth.

In some embodiments, the slider includes one or more wall, which when adjacent to dental features being scanned, guides movement of the slider along the dental features e.g., along a jaw. In some embodiments, the wall extends in a direction from a distal (optionally elongate) portion of the add-on towards dental features. For example, in a direction including a component perpendicular to a direction of elongation of the distal portion of the add-on. In some embodiments, the wall extends along a length of the distal portion, for example, by a length of 0.1-2 cm, or 0.5-2 cm, or about 1 cm, or lower or higher or intermediate lengths or ranges. In some embodiments, the slider includes more than one wall, for example, two side walls both extending towards dental features from the distal portion and extending along the distal portion towards a body of the add-on and/or towards the smartphone. Where the two side walls establish, in some embodiments, a cavity sized and/or shaped to receive dental feature/s e.g., to guide movement of the slider and/or add-on during scanning (e.g., self-scanning)

Potentially, such size and/or shape of the add-on enables swiping scanning movement/s.

In some embodiments, the slider includes one or more optical element for directing light between dental feature/s (e.g., within the cavity of the slider) and a body of the add-on. For example, one or more optical element for transferring light from dental feature/s to the body and/or one or more optical element for transferring light from the body to the dental feature/s. In some embodiments, the slider includes one or more optical element configured to split a FOV of an optical element.

In some embodiments, the optical path of the add-on includes directing one or more Field of View (FOV) (e.g., of imager/s of the smartphone or IOS) and/or light (e.g., structured light) towards more than one surface of a tooth or teeth. For example, more than one of occlusal, lingual, and buccal surfaces of a tooth or teeth. In some embodiments, the optical path is provided by one or more optical element of the add-on (e.g., hosted within an add-on housing). Where, in some embodiments, the add-on includes one or more mirrors which transfer (e.g., change a path of) light. Where, in some embodiments, one or more mirrors are located in a distal portion of the add-on e.g., the slider.

Potentially, this multi-view optical path enables scanning of a plurality of tooth surfaces e.g., for a given position of the add-on and/or in a single movement. For example, where the optical path of the add-on provides light transfer from occlusal, lingual, and buccal surfaces, a user moving the add-on along a dental arc, in some embodiments, potentially collects images of all tooth surfaces of the arc in the movement.

In some embodiments, optical path portion/s of the add-on local to the dental features being scanned have adjustable angle with respect to the add-on body. In some embodiments, an angle of a portion of the add-on local to the dental features being scanned and/or forming an end of the optical path of the add-on changes (e.g., moving about a joint) with respect to the body of the add-on and/or to the smartphone. Potentially enabling scanning of the dental arc, with associated changes in orientation of teeth with respect to the mouth opening while changing an angle of the add-on body and/or smartphone with respect to the mouth to a lesser degree than the portion of the add-on local to the dental features.

An aspect of some embodiments relate to an add-on including a single optical element which configures light provided by the smart-phone for illumination of dental features for scanning. In some embodiments, the optical element includes optical power for focusing of the light and one or more patterning element which prevents transmission of portion/s of light emitted.

A broad aspect of some embodiments relate to correcting scan data collected using the add-on (or using an intra oral scanner). In some embodiments, one or more image collected from a distance to dental feature/s is used. For example, from outside the oral cavity, e.g., at a separation of at least 1 cm, or 5 cm or lower or higher or intermediate separations, from an opening of the mouth. In some embodiments, the images used for generating the 3D model have a smaller FOV than that of the 2D images of larger FOV e.g., where the 2D image FOV is at least 10%, or at least 50% or at least double, or triple, or 1.5-10 times a size of the FOV of one or more of the images used to generate the 3D model. In some embodiments, correction is using a 2D image (e.g., as opposed to a 3D model) where, in some embodiments, the 2D image is collected using a smartphone. For example, in some embodiments, a user self-scanning performs a scan using the add-on and also collects one or more picture (e.g., using a smartphone camera) of dental feature/s, the pictures then being used to correct error/s in the scan data. For example, accumulated errors associated with stitching of images to generate a 3D model. In some embodiments, the 3D model is generated using acquired images of dental features illuminated with patterned light (also herein termed “structured” light).

In some embodiments, the 2D images are acquired as a video recording e.g., using a smartphone video recording feature. In some embodiments, at least two 2D images, taken separately and/or inside a video, are used to generate a 3D model of the dental features using stereo. In some embodiments, more than one 2D image (e.g., 2 or more) potentially increase accuracy of correction of 3D model using a 2D image e.g., correction as described above. In some embodiments, additional image/s (e.g., more than one 2D image) are used to increase depth accuracy which, in some embodiments, is reduced and/or low associated with correcting a 3D model using a single 2D image. In some embodiments, the additional image/s are used to verify accuracy of a final 3D model, the image/s used to test accuracy of fitting the projected 2D image of the obtained 3D model to acquired 2D images.

An aspect of some embodiments relate to monitoring of a subject using follow-up scan data which, in some embodiments, is acquired by self-scanning. In some embodiments, a detailed initial scan (or more than one initial scan) is used along with follow-up scan data to monitor a subject. In some embodiments, the initial scan being updated using the follow-up scan and/or the follow-up scan being compared to the initial scan to monitor the subject.

A broad aspect of some embodiments relate to adapting an electronic communication device and/or a handheld electronic device (e.g., smartphone) for intraoral scanning. Where intraoral scanning, in some embodiments, includes collecting one or more optical measurement (e.g., image) of dental feature/s and, optionally, other dental measurements. In some embodiments, an add-on is connected to the smartphone, for example, enabling one or more feature of the smartphone to be used for intraoral scanning e.g., within a subject's mouth.

An aspect of some embodiments relates to an add-on device which adapts one or more optical element of the smartphone for dental imaging. Optical elements including, for example, one or more imager and/or illuminator.

In some embodiments, adapting of optical elements includes transferring a FOV of the optical element (or at least a portion of the FOV of the optical element) to a different position.

In this document, regarding imagers and/or imaging, description is of transfer of the FOV of the imager through an optical path of the add-on. However, it should be understood that this refers to an optical path through the add-on providing light emanating from a FOV region (e.g., outside the add-on) to the imager. In some embodiments, the light includes light emanating from (e.g., reflected by) one or more internal surface within the add-on.

In some embodiments, the FOV region and/or a portion of an add-on is positioned within and/or inside a subject's mouth and/or oral cavity e.g., during scanning with the add-on and smartphone. Where, in some embodiments, positioning is within a space defined by a dental arch of one or more of the subject's jaws. An imaging FOV and/or images acquired with the add-on for example, including lingual region/s of one or more dental feature (e.g., tooth and/or dental prosthetic) and/or buccal region/s of dental feature/s e.g., the features including pre-molars and/or molars. In some embodiments, add-on is used to scan soft tissue of the oral cavity.

In some embodiments, the add-on moves a FOV of one or more imager and/or one or more illuminator away from a body of the smartphone. For example, by 1-10 cm, in one or more direction, or lower or higher or intermediate ranges or distances. For example, by 1-10 cm in a first direction, and by less than 3 cm, or less than 2 cm, or lower or higher or intermediate distances, in other directions.

In some embodiments, the add-on, once attached to the smartphone extends (e.g., a central longitudinal axis of the add-on e.g., elongate add-on body) in a parallel direction (or at most 10 or 20 or 30 degrees from parallel) to one or both faces of the smartphone.

In some embodiments, the add-on, once attached to the smartphone extends (e.g., a central longitudinal axis of the add-on e.g., elongate add-on body) in a perpendicular direction (or at or at most 10 or 20 or 30 degrees from perpendicular) to one or both faces of the smartphone. A potential benefit being case of viewing of the smartphone screen. For example, directly e.g., where the add-on extends from a screen face of the smartphone. For example, indirectly e.g., via a mirror generally opposite the subject.

In some embodiments, an angle of extension is between perpendicular and parallel. For example, an angle of extension of the add-on from the smartphone of 30-90 degrees.

Where the add-on moves and/or transfers the FOV/s, for example, in a direction (e.g., a direction of a central longitudinal axis of the add-on body) generally parallel (e.g., within 5, or 10, or 20 degrees of parallel) to a front and/or back face of the smartphone. Where, in some embodiments, the front face hosts a smartphone screen and the back face hosts one or more optical element of the smartphone (e.g., imager, e.g., illuminator). In some embodiments, a smallest cuboid shape enclosing outer surfaces of the smartphone defines faces and edges of the smartphone. Where, in some embodiments, faces are defined as two opposing largest sides of the cuboid and edges are the remaining 4 sides of the cuboid.

Where in some embodiments, including perpendicular, parallel and between perpendicular and parallel directions of extension of the add-on elongate body from an orientation the smartphone face, FOVs emanating from the add-on body (e.g., imaging and/or illuminating) are perpendicular from the longitudinal axis of the add-on body (or at most 10 degrees, or 20 degrees, or 30 degrees from perpendicular).

Where in some embodiments, including perpendicular, parallel and between perpendicular and parallel directions of extension of the add-on elongate body from an orientation the smartphone face, FOVs emanating from the add-on body (e.g., imaging and/or illuminating) are parallel from the longitudinal axis of the add-on body (or at most 10 degrees, or 20 degrees, or 30 degrees from perpendicular). For example, extending from a distal tip of the add-on body.

In some embodiments, at least a portion (e.g., a body of the add-on) of the add-on is sized and/or shaped for insertion into a human mouth. One or more FOV, in some embodiments, emanating from this portion.

In some embodiments, transfer is by one or more transfer optical element, the element/s including mirror/s and/or prism/s and/or optical fiber. In some embodiments, one or more of the transfer element/s has optical power, e.g., a mirror optical element has a curvature.

In some embodiments, transfer is through an optical path, and the add-on includes one or more optical path for one or more device optical element e.g., smartphone imager/s and/or illuminator/s. In some embodiments, optical path/s pass through a body of the add-on. In some embodiments, transfer of FOV/s includes shifting a point and/or region of emanation of the FOV from a body of the smartphone to a body of the add-on.

In some embodiments, FOV/s of illuminator/s are adjusted for dental imaging. Where, in some embodiments, one or more of illumination intensity, illuminator color, illumination extent are selected and/or adjusted for dental imaging.

In some embodiments, an add-on illuminator optical path includes a patterning element. Where, for example, an optical path for light emanating from an illuminator of the smartphone (and/or from an illuminator of the add-on) patterns light emanating from the add-on. Alternatively, or additionally, in some embodiments, an illuminator is configured to directly illuminate with patterned light e.g., where the smartphone screen is used as an illuminator.

In some embodiments, the patterned light incident on dental feature/s (e.g., when the add-on is at least partially inserted into a mouth) is suitable to assist in extraction of geometry (e.g., 3D geometry) of the dental feature/s from images of the dental features lit with the patterned light. Where, for example, in some embodiments, separation between patterning elements (e.g., lines of a grid) is 0.1-3 mm, or 0.5-3 mm, or 0.5 mm-1 mm, or lower or higher or intermediate separations or ranges, when the light is incident on a surface between 0.5-5 cm, or 0.5-2 cm, or lower or higher or intermediate distances or ranges, from a surface of the add-on from which the FOV emanates.

In some embodiments, the illuminator optical path includes one or more additional element having optical power, for example, one or more lens and/or prism and/or curved mirror. Where, for example, in some embodiments, element/s having optical power adjust the projected light FOV to be suitable for dental imaging with the add-on. For example, in some embodiments, an angle of a projection FOV is adjusted to overlap with one or more imaging FOV (alternatively or additionally, in some embodiments, an imaging FOV is adjusted to overlap with one or more illumination FOV). In some embodiments, projected light is focused by one or more lens.

In some embodiments, an imager FOV for one or imager is adjusted by the add-on, e.g., by one or more optical element optionally including optical elements having optical power e.g., mirror, prism, optical fiber, lens. Where adjusting includes, for example, one or more of; transferring, focusing, and splitting of the FOV.

In some embodiments, performance and/or operation of device optical element/s of the smartphone are adapted for intraoral scanning. For example, in some embodiments, optical parameter/s of one or more optical element are adjusted. For example, by software installed on the smartphone, the software interfacing with smartphone control of the optical elements.

In some embodiments, scanning includes collecting images of dental features using one or more imager e.g., imager of the smartphone. Where, in some embodiments, the imager acquires images through the add-on (e.g., through the optical path of the add-on).

In some embodiments, one or more imager imaging parameter (e.g., of the smartphone) is controlled and/or adjusted e.g., for intraoral scanning. For example, position of emanation and/or orientation of an imaging FOV. For example, in some embodiments, one or more of imager focal distance and frame rate are selected and/or adjusted for dental scanning. For example, in some embodiments, a subset of sensing pixels (e.g., corresponding to a dental region of interest ROI) are selected for image acquisition. For example, in some embodiments, zoom of one or more smartphone imager is controlled. For example, to maximize a proportion of the FOV of the imager which includes dental feature/s and/or calibration information.

In some embodiments, one or more parameter of one or more illuminator e.g., of the smartphone is adjusted and/or controlled. For example, one or more of; when one or more illuminator is turned on, which portion/s of an illuminator are illuminated (e.g., in a multi-LED illuminator which LEDs are activated), color of illumination, power of illumination.

In some embodiments, during acquisition of images, at least a portion of the add-on is inserted into the subject's mouth for example, potentially enabling collection of images of inner dental surfaces. In some embodiments, e.g., where the add-on remains outside the mouth, one or more mirror positioned within the mouth enables imaging of inner dental regions.

In some embodiments, one or more fiducial is used during scanning and/or calibration of the add-on connected to the smartphone.

Where, in some embodiments, fiducial/s are attached to the user. In some embodiments, the fiducial is positioned in a known position with respect to dental feature/s. For example, by attachment directly and/or indirectly to rigid dental structures e.g., attachment to a tooth e.g., attachment by a user biting down on a biter connected to the fiducial/s.

In some embodiments, the fiducials are used in calibration of scanned images e.g., where fiducial/s of known color and/or size and/or position (e.g., position with respect to the add-on and/or smartphone) are used to calibrate these features in one or more image and/or between images.

In some embodiments, a check retractor is used during scanning, for example, to reveal outer surfaces of teeth. In some embodiments, the check retractor includes one or more fiducial and/or mirror e.g., positioned within the oral cavity.

A broad aspect of some embodiments relate to a user performing a self-scan (e.g., dental self-scan) using an add-on and a smartphone (e.g., the user's smartphone).

In some embodiments, the user is guided during scanning. For example, by one or more communication through a smartphone user interface. For example, by aural cues e.g., broadcast by smartphone speaker/s. For example, by one or more image displayed on the smartphone screen. Where, in some embodiments, while a portion of the add-on is within the user's mouth, the user views the image/s displayed on the smartphone screen.

In some embodiments, when the add-on attached to the smartphone is used for scanning, the smartphone is orientated so that the user can directly view the smartphone screen. Where, for example, the add on extends into the mouth from a lower portion of a front face of the smartphone, e.g., a central longitudinal axis of the add-on being about perpendicular, or within 20-50 degrees of perpendicular to a plane of the smartphone screen and/or front face.

In some embodiments, when the add-on attached to the smartphone is used for scanning, the smartphone screen is orientated away from the user and the user views the screen in a reflection of the screen in a mirror. For example, an external mirror e.g., opposite to the user e.g., mirror on a wall.

In some embodiments, the add-on includes one or more mirror angled with respect to the smartphone screen and user's viewpoint to reflect at least a portion of the smartphone screen towards the user.

In some embodiments, display to a user is 3D, where, in some embodiments different colored display on the smartphone screen is selected to produce a 3D image when the user is wearing a corresponding pair of glasses. For example, red/cyan 3D image production.

In some embodiments, displayed images are focused so that the image plane is not at the smartphone screen. For example, where the screen (and/or reflection of the screen) is close to the user, placing the image plane at a more comfortable viewing distance e.g., further away from the user than the smartphone screen.

In some embodiments, dental scanning using the add-on and a smartphone is performed by a subject themselves e.g., at home. Where, in some embodiments, collected measurement data is processed and/or shared for example, to provide monitoring (e.g., to a healthcare professional) and/or to provide feedback to the subject. The subject self-scanning potentially enables monitoring and/or treatment of the subject more frequently than that provided by in-office dental visits and/or imaging using a standard intraoral scanner.

In some embodiments, dental scanning using the add-on and a smartphone is performed, for example, by a user (e.g., at home and/or without the user having an in-person appointment with a healthcare professional) is performed periodically e.g., to monitor the subject. Where, in some embodiments, the scanning data is reviewed, for example, by a healthcare professional. In some embodiments, scanning and/or monitoring is of one or more of; oral cancer, gingivitis, gum inflammation, cavity/ies, dental decay, plaque, calculus, tipping of teeth, teeth grinding, erosion, orthodontic treatment (e.g., alignment with aligner/s), teeth whitening, tooth-brushing.

In some embodiments, scan data is used as an input to an AI based oral care recommendation engine. Where the engine, in some embodiments, outputs instructions and/or recommendations (e.g., which are communicated to the subject), based on the scan data e.g., one scan and/or periodic scan data over time.

An aspect of some embodiments relate to an add-on for a smartphone (or other electronic device e.g., IOS) which includes a probe. Where, in some embodiments, the probe is sized and/or shaped to be placed between teeth and/or between a tooth surface and gums and/or into a periodontal pocket. In some embodiments, the probe extends away from a body of the add-on. In some embodiments, the probe is visible in at least one FOV of the electronic device imager. In some embodiments, known position of the probe (e.g., a tip of the probe) with respect to one or more portion of the add-on is used in measurement of dental feature/s and/or in processing of images of dental features acquired. In some embodiments, the probe includes one or more marking. In some embodiments, markings have a known spatial relationship with respect to each other. In some embodiments, the spatial positioning of one or more marking is known with respect to one or more other portion of the add-on. In some embodiments the probe includes a light source e.g., located at and/or where light from the light source emanates from a tip of the probe. In some embodiments, the light source provides illumination for transilluminance measurements. In some embodiments, the light source is located proximal (e.g., closer to and/or within a body of the add-on) of the probe tip and the light is transferred to the tip e.g., by fiber optic.

An aspect of some embodiments of the disclosure relates to using an add-on having a distal portion sized and/or shaped for insertion into the mouth to expose region/s of the mouth to infrared (IR) light. Where, in some embodiments, dental surface/s are exposed to IR light, for example, as a treatment e.g., for bone generation. A potential advantage of using an add-on is the ability to access dental surfaces and deliver light to them. In some embodiments, IR light is used to charge power source/s for device/s within the mouth, for example, batteries for self-aligning braces and the like.

Throughout this document the term “smartphone” has been used, however this term, for some embodiments, should be understood to also refer to and encompass other electronic devices, e.g., electronic communication devices, for example, handheld electronic devices, e.g., tablets, watches.

Before explaining at least one embodiment of at least one of the inventions disclosed herein in detail, it is to be understood that such inventions are not necessarily limited in its application to the details of construction and the arrangement of the components and/or methods set forth in the following description and/or illustrated in the drawings and/or the Examples of the described embodiments. Such inventions are capable of other embodiments or of being practiced or carried out in various ways.

### Exemplary System

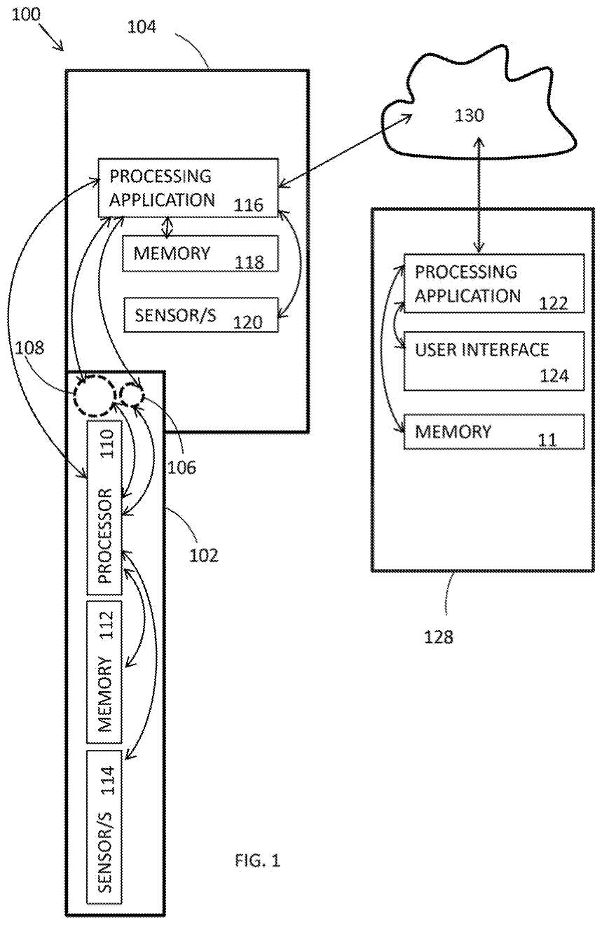

FIG. 1 is a simplified schematic of a dental measurement system 100, according to some embodiments.

In some embodiments, system 100 includes a smartphone 104 attached to an add-on 102. Where add-on 102 has one or more feature of add-ons as described elsewhere in this document.

Alternatively, in some embodiments, element 104 is a device including an imager e.g., an intraoral scanner (IOS) 104. Description regarding element 104 should be understood to refer to a smartphone and an IOS.

In some embodiments, add-on 102 is mechanically connected to smartphone 104. In some embodiments, optical elements 108, 106 of the smartphone are aligned with optical pathways of add-on 102.

In some embodiments, smartphone 104 includes a processing application 116 (e.g., hosted by a processor of the smartphone) which controls one or more optical element 108, 106 of the smartphone (e.g., imager and/or illuminator) and/or receives data from the element/s e.g., images collected by imager 108.

In some embodiments, processing application 116 stores collected images in a memory 118 and/or uses instructions and/or in memory in processing of the data. For example, in some embodiments, previous scan data is stored in memory 118 is used to evaluate a current scan. In some embodiments, one or more additional sensor 120 is connected to processing application 116 receiving control signals and/or sending sensor data to the processing application. For example, in some embodiments, Inertial Measurement Unit (IMU) measurements are used in evaluating and/or processing collected images. For example, in some embodiments, illumination and/or imaging is carried out by additional optical elements of the smartphone which, for example, are not optically connected to the add-on.

Optionally, in some embodiments, the add-on includes a processor 110 and/or a memory 112 and/or sensor/s 114. In some embodiments, add-on sensor/s include one or more imager. In some embodiments, processor 110 has a data connection to the smartphone processing application 116.

In some embodiments, the smartphone is connected to other device/s 128 e.g., via the cloud 130. In some embodiments, processing of data (e.g., generation of 3D model/s using collected images and optionally other data) is performed in the cloud. In some embodiments, it is performed by one or more other device 128. For example, at a dental surgery, for example, a dental practitioner's device 128. Where, in some embodiments, inputted instructions via a user interface 124 are transmitted to the subject's smartphone 104 e.g., to control and/or adjust scanning and/or interact with the subject.

In some embodiments, add-on 102 is connected to smartphone 104 through a cable (e.g., with a USBC connector). In some embodiments, add-on 102 is wirelessly connected to smartphone 104 (e.g., Wi-Fi, Bluetooth). In some embodiments, add-on 102 is not directly mechanically connected to smartphone 104 and/or not rigidly connected to smartphone 104 (e.g., only connected by cable/s).

In some embodiments, system 100 includes one or more additional imager (not illustrated). For example, connected wirelessly to the smartphone and/or cloud. Where, for example, in some embodiments, sensor/s 114 of add-on 102 include one or more imager, also herein termed a “stand-alone camera”.

In some embodiments, the system is configured to receive feedback from users on function and/or aesthetics and/or suggestion/s for treatments and/or other uses.

In some embodiments, a mobile electronic device is not used. Where, for example a system includes at least one imager configured to be inserted into a mouth, optionally one or more illuminator (e.g., including one or more pattern projector) configured to illuminate dental surfaces being imaged by the at least one imager. Where, in some embodiments, data is processed locally, and/or by another processor (e.g., in the cloud). In some embodiments, the imager and pattern projector are housed in a device including one or more feature of add-ons as described within this document, but where the smartphone is absent, device including access to power data connectivity and one or more imager.

### Exemplary Methods

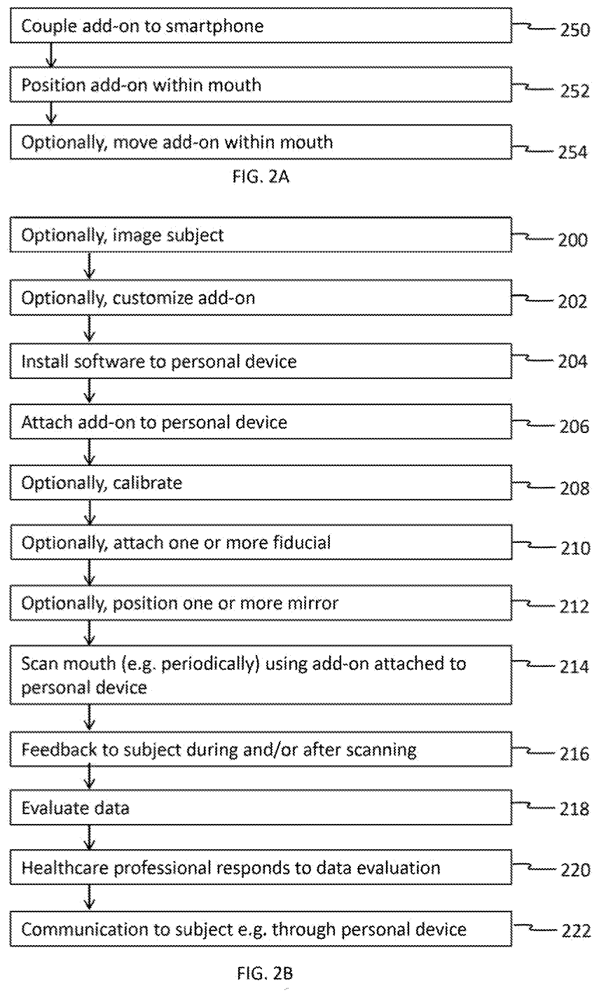

FIG. 2A is a method of intraoral scanning, according to some embodiments.

At 250, in some embodiments, an add-on is coupled to a smartphone. For example, connected mechanically (e.g., as described elsewhere in this document). For example, additionally or alternatively to a mechanical connection, data connected (e.g., as described elsewhere in this document)

In some embodiments, coupling is by placing at least a portion of the smartphone into a lumen of the add-on. Where, in some embodiments, the lumen is sized and/or shaped to fit the smartphone sufficiently closely that friction between the smartphone and the add-on holds the smartphone in position with respect to the add-on. In some embodiments, add-on lumen is flexible and/or clastic, deformation (e.g., clastic deformation) of the add-on acting to hold the add-on and smartphone together.

Additionally, or alternatively, in some embodiments, coupling includes adhering (e.g., glue, Velcro) and/or using one or more connector e.g., connector/s wrapped around the add-on and smartphone. Additionally, or alternatively, in some embodiments, coupling includes one or more interference fit (e.g., snap-together) and/or magnetic connection.

At 252, in some embodiments, at least a portion of the add-on is positioned within the subject's mouth. For example, by the subject themselves. In some embodiments, an edge and/or end of the add-on is put into the mouth. In some embodiments, only the add-on enters the oral cavity and the smartphone remains outside. Alternatively, in some embodiments, a portion of the smartphone enters the oral cavity e.g., an edge and/or corner of the smartphone e.g., which is attached to the add-on.

At 254, in some embodiments, the add-on is moved within the subject's mouth. For example, by the subject e.g., where, in some embodiments, the subject moves the add-on according to previously received instructions and/or instructions and/or prompts communicated to the subject e.g., via one or more user interface of the smartphone.

In some embodiments, a user moves the periscope inside the mouth e.g., in swipes. Where, in some embodiments, swipe movement includes a movement in a single direction along a dental arch e.g., without rotations. Where a potential advantage of swipe movement/s is case of performance by the user e.g., when self-scanning.

In some embodiments, exemplary scanning (e.g., where a user is instructed to perform the scanning) includes, for the upper dental arch (e.g., where the tongue is less likely to interfere), one or more swipe e.g., two swipes one for each half of the upper dental arch.

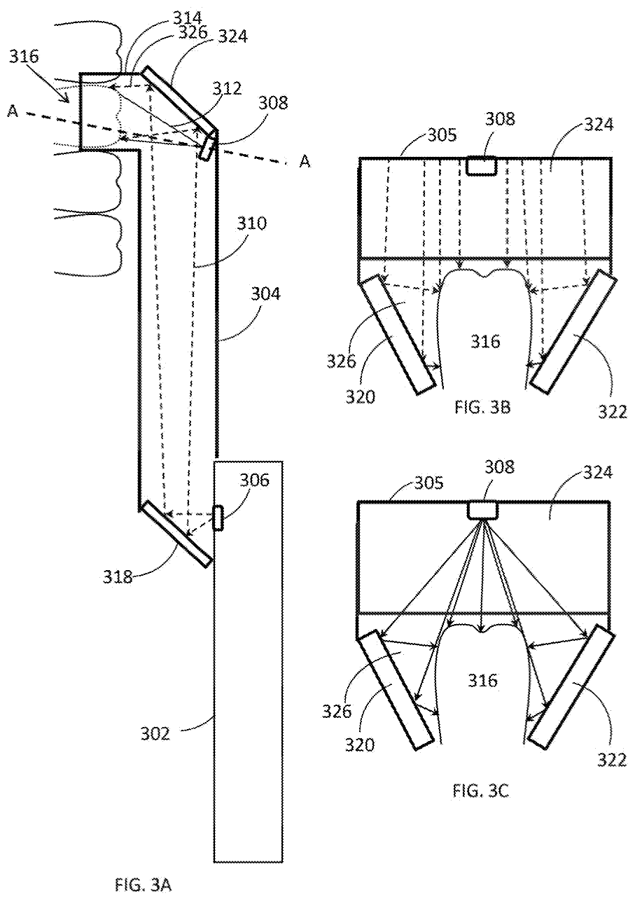

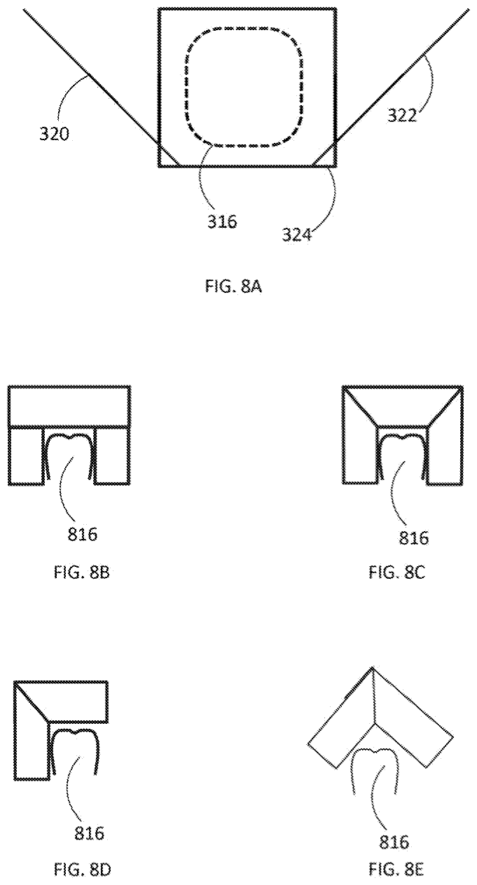

In some embodiments, two views of dental features are provided to the imager by the add-on e.g., referring to FIG. 3B and FIG. 3C e.g., where the add-on includes one of mirrors 320, 322. For example, as illustrated in FIG. 8D. In some embodiments, an upper dental arch scan, where the tongue is less likely to interfere with scanning, the user scans the teeth from the buccal side, for example with 2 swipes, one for each side of the upper jaw e.g., collecting images of buccal and occlusal sides of the teeth. In some embodiments, inner (lingual) part of the upper jaw is scanned e.g., in a single or e.g., in two swipes, one for each side (left/right) of the upper jaw. In some embodiments, performing lingual swipe scanning after the buccal swipe scanning enables removal of soft tissue e.g., lips and/or check from image/s collected and/or a 3D model generated using the images. In some embodiments, lip/s and/or inner check tissue appear behind teeth, and/or touching the buccal part of teeth during lingual scanning. In some embodiments, lip/s and/or check/s are removed from the 3D model during stitching of lingual and buccal side images by selecting views for particular shared portion/s of images. For example, where occlusal portions of the 3D model of the teeth, which appear in both lingual and buccal scans are provided by one of the scans where the add-on is mechanically holding the check/s and/or lips away from the occlusal surface/s of the teeth.

In some embodiments, when scanning the lower dental arch lingual swipes, in some embodiments, capture the tongue behind the teeth. In some embodiments, the tongue is removed from images and/or the 3D model using knowledge that the tongue is located lingual to the tooth/teeth and using color segmentation to separate between white tooth/teeth from red/pink gums and tongue and depth information (e.g., from patterned light).

In some embodiments, image/s of the bite are acquired and used to align 3D models of the upper and lower dental arches to give bite information. In some embodiments, the image/s are collected from one side of the dental arch only e.g., lingual or buccal and/or from a portion of the mouth. For example, using bite swipe/s (e.g., at least two). In some embodiments, bite swipe/s and/or image/s (e.g., using smartphone camera directly and not through an add-on) collected from outside the mouth. In some embodiments, bite scan information is only of a portion of the dental arches, for example 3 teeth on one side of 3 teeth on each right/left side which, in some embodiments, is enough for bite alignment e.g., of the 3D arch models.

In some embodiments, splitting of FOVs of the imager enables scanning in fewer swipes. For example, using a scanner that can capture a single side of tooth in a single jaw, will, in some embodiments, use 3 swipes to capture one side of one jaw. Corresponding, for example, to up to 12 swipes to capture a full mouth.

Using a scanner as described in FIG. 8D, in some embodiments, uses only 2 swipes per jaw, per side corresponding to, for example, up to 8 swipes per mouth.

Using a scanner as described in FIG. 3A, for example, uses one swipe for one side of each jaw corresponding to, for example, up to 4 swipes per mouth.

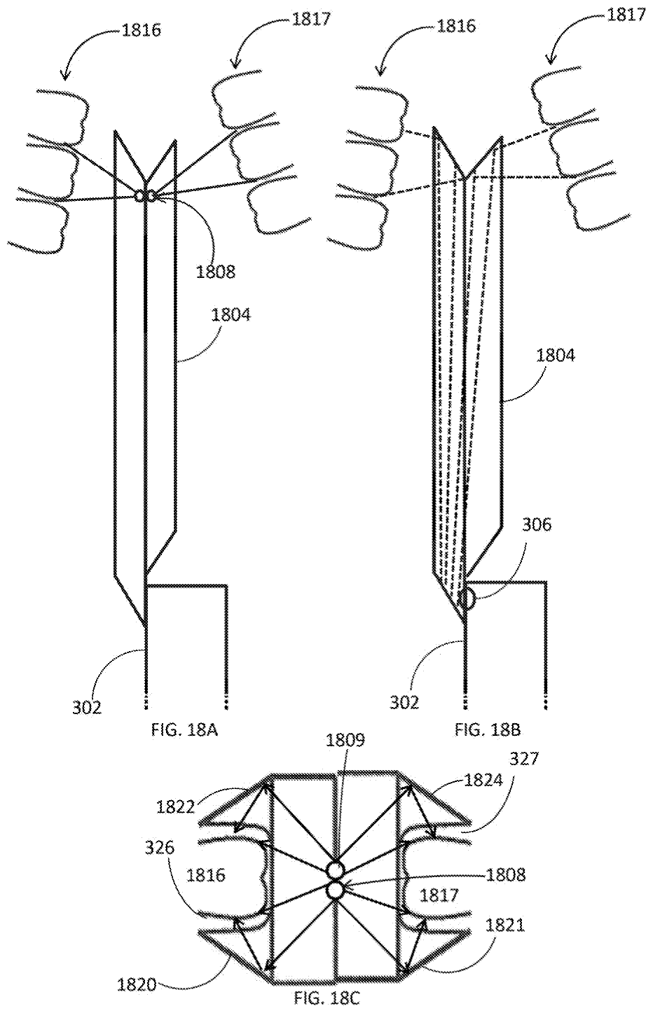

Using the scanner described at FIGS. 18A-B in some embodiments, reduces the number of swipes for a full mouth (2 jaws) to 2 (right side of mouth and left side of mouth).

Reducing the number of used swipes has a potential advantages of case and/or increased likelihood of high quality of results for, for example, self-scanning. For example, assuming that each swipe has 90 percent success rate and the full mouth scan successes rate is 0.9 times the number of swipes.

In some embodiments, reducing a number of swipes to capture the full mouth to a single swipe is by the user rotating the add-on (e.g., without lifting and/or removing the add-on from the mouth when the add-on reaches the front teeth. For example, a user starting scanning from the back side of the right side of the mouth, moving from back side to the front, rotating the IOS when it reaches the front tooth and continue scanning of the left side of the mouth while moving from the front teeth to the back area of the left side.

FIG. 2B is a flowchart of a method, according to some embodiments.

At 200, optionally, in some embodiments, the subject is imaged. For example, using one or more type of imaging e.g., x-ray, MRI, ultra-sound. In some embodiments, the subject is imaged using an intraoral scanner e.g., a commercial dental intraoral scanner e.g., where scanning is by a healthcare professional. In some embodiments, the subject is imaged by a healthcare professional using an add-on and a smartphone e.g., the subject's smartphone. For example, to collect initial scan data. For example, as part of training the subject in self-scanning using the add-on. In some embodiments, imaging data (e.g., from one or more data source) is used to generate a model (e.g., 3D model) of oral feature/s inside the mouth for the subject. At 202, optionally, in some embodiments, the add-on is customized.

In some embodiments, an add-on is customized and/or designed and/or adjusted to fit smartphone mechanical dimensions and/or optics (e.g., imager/s and/or illuminator/s (e.g., LED/s)) positions.

In some embodiments, customizing includes selecting relative position of optical pathways of the add-on and/or connection and/or connectors of the add-on. Where, in some embodiments, selecting is based on position and/or size of smartphone feature/s e.g., of the smartphone to be used in performing the scanning. Where feature/s include, for example, one or more of smartphone camera size and/or position on the smartphone, smartphone illuminator size and/or position on the smartphone, smartphone external (e.g., of the smartphone body) dimension/s, smartphone display size and/or position. In some embodiments, selecting is additionally or alternatively based on smartphone camera and/or illuminator and/or screen features e.g., camera resolution; number of pixels, pixel size, sensitivity, focal distance, illuminator; power, field of view, color of illuminating light.

In some embodiments, customizing includes adjusting one or more portion of the add-on e.g., based on a model of the subject's smartphone. Where, in some embodiments, the adjustment is performed when the subject receives the add-on (e.g., by a health practitioner), and/or the subject themselves adjusts the add-on.

In some embodiments, adjustment includes aligning optical pathway/s of the add-on to one or more camera and or one or more illuminator of the smartphone. In some embodiments, aligning includes moving relative position of a proximal end of the add-on, and/or moving position of one or more portion of a proximal end of the add-on, for example, with respect to other portion/s of the add-on.

In some embodiments, customization includes selecting a suitable add-on. Where, for example, a kit includes a plurality of different add-ons suitable for use with different smart phones. In some embodiments, customizing includes combining add-on portions. For example, in some embodiments, an add-on is customized by selecting a plurality of parts and connecting them together to provide an add-on. Where, in some embodiments, customization is of the parts and/or of how the parts are connected.

For example, in some embodiments, an add-on proximal portion is selected from a plurality of proximal portions for example, for connecting to a distal portion to provide an add-on for a subject.

In some embodiments, customizing includes manufacture of the add-on e.g., for different smartphones. For example, an individually customized add-on e.g., for a specific user.

In some embodiments, portion/s of an add-on and/or a body of an add on are printed using a 3D printer e.g., in printed plastic.

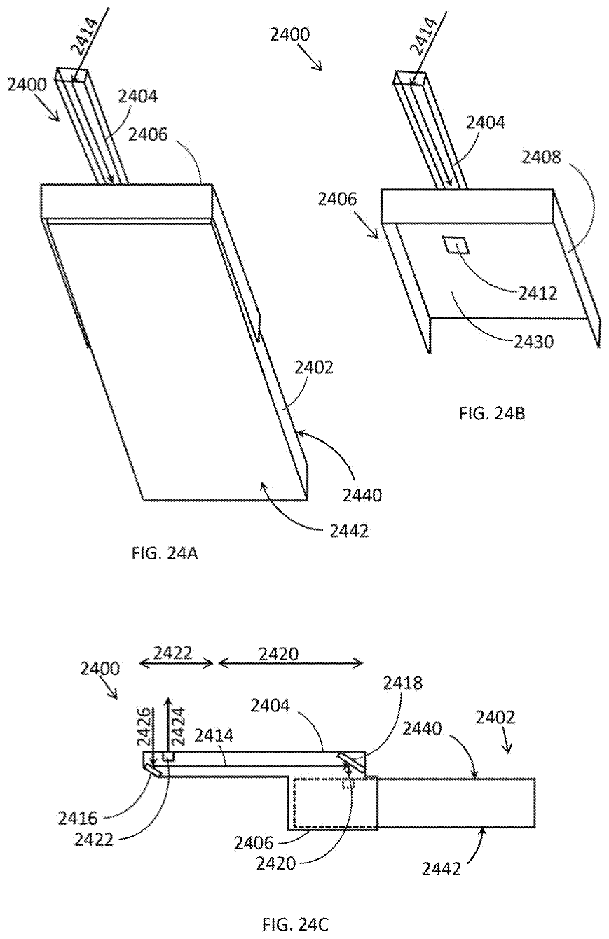

In some embodiments, an add-on includes two or more parts. For example, in some embodiments, a part (e.g., portion 2422 FIG. 24C) is manufactured using mass production methods (e.g., plastic injection molding) and a second part (e.g., portion 2420 FIG. 24C) is customized for the user e.g., the user's smartphone. Where the second part, in some embodiments, is manufactured 3D printing.

For example, in an exemplary embodiment, a first portion of the add-on an elongate and/or distal portion of an add-on, including at least one mirror and, in some embodiments, at least one pattern projector. In some embodiments, a second portion of the add-on, includes optical element/s to align an imager of the smartphone to the optical path. Where, in some embodiments, the first portion is mass produced to be attached to the second portion which, in some embodiments, is customized for a user and/or smartphone model e.g., using 3D printing.

In some embodiments, an add-on is customized using subject data which is for example, received via a smartphone application. Where, in some embodiments, subject data includes one or more of; smartphone data and medical and/or personal records. For example, based on one or more of; a smartphone model, subject sex and/or age, the type of scanning to be performed. In some embodiments, the add-on is customized according to user personalization e.g., a user selects one or more personalization e.g., via a smartphone application. In some embodiments, optical elements e.g., mirror/s and/or lenses are the same for personalized add-ons e.g., potentially reducing a number of bill of materials (BOM) parts and/or simplifying manufacture and/or an assembly line for manufacture of personalized add-ons. Where assembly of a personalized add-on, in some embodiments, is by constructing (e.g., by 3D printing) an add-on body based on the user requirements and adding a same projector and/or mirror parts.

At 204, in some embodiments, software is installed on a personal device (e.g., smartphone) to be used in dental scanning. For example, an application is downloaded onto the users smartphone.

In some embodiments, the software sends the smartphone model and/or feature/s including imager feature/s and/or illuminator feature/s (e.g., relative position, optical characteristic/s) of the smartphone and/or additional details (e.g., including one or more detail inputted by a user) are sent by the software to a customization center. Where, in some embodiments, an adaptor is customized according to the received details. For example, by 3D printing. In some embodiments, customized/s portions of an add-on are combined with standard portions to produce an add-on. Where, in some embodiments, the combining is performed at production or by the user who receives the parts separately and attaches them. Once customized the add-on and/or add-on is provided to the user.

In some embodiments, the application receives user inputs and/or outputs instructions to the user e.g., reminders to scan, instructions before and/or during scanning.

In some embodiments, the application interfaces with smartphone hardware to control imaging using one or more imager of the smartphone and/or illumination with one or more illuminator of the smartphone. Illuminators, in some embodiments, including the smartphone screen.

In some embodiments, acquisition and/or processing of acquired images is controlled. For example, in some embodiments, light transferred through the add-on optical path (e.g., through reflection at one or more mirrors) is incident on less than all of the pixels of a digital (e.g., CMOS) imager (e.g., of the smartphone) and/or useful data is incident on less than all of the pixels. In some embodiments, software confines imaging to a ROI (Region of interest) where only the ROI within the imager FOV is captured, and/or processed and/or saved. Potentially enabling a higher frame rate (e.g., frames per second FPS) of imaging and/or a shorter scanning time. In some embodiments, imaging is confined to more than one region of the FOV, for example, a region for each FOV where the imager FOV is split into more than one region (e.g., splitting as described elsewhere in this document).

In some embodiments, zoom of a smartphone imager is controlled, by controlling zoom optically (e.g., by controlling optics of the imager), and/or digitally. In some embodiments, zoom is controlled to maximize a proportion of image pixels which include relevant information. For example, which include dental features and/or calibration target/s.

In some embodiments, exposure time of the smartphone imager is controlled. For example, to align exposure time to frequency of illumination source/s e.g., potentially reducing flickering and/or banding. For example, in some embodiments, exposure time and additional features of the smart phone camera are adjusted to remove the ambient flickering effect e.g., to 50 Hz, 60 Hz, 100 Hz and 120 Hz.

In some embodiments, the application changes smartphone software control of one or more imager and/or illuminator.

For example, in some embodiments, one or more automatic control feature is adjusted and/or turned off and/or compensated for. Where compensation includes, for example, during processing of images to acquire depth and/or other data for generation of a model of dental features, compensating for changes to images associated with the automatic control feature. Where compensation includes, for example, prior to processing of images to acquire data regarding dental features, compensating for change/s to the images associated with the automatic control feature.

Where automatic feature/s which are disabled and/or adjusted and/or compensated for are one or more of those which affect calibration of imaging for capture of images from which depth information is extractable. For example, automatic control feature/s which affect one or more of color, sharpness, frame rate. For example, one or more, image signal processing and/or AI imaging feature as controlled and/or implemented by a smartphone processing unit (e.g., processing application 116 FIG. 1).

In some embodiments, optical image stabilization (OIS) is controlled by the application. OIS, generally, involves adjusting position of the optical component/s, for example, the image sensor/s (e.g., CMOS image sensor) and/or lens/s. For example, to create smoother video (e.g., despite vibration of the smartphone). In some embodiments, OIS affects processing of images requiring known position of feature/s (e.g., position of patterned light) within the imager FOV and/or within an acquired image. In some embodiments, OIS software is disabled (at least partially) potentially increasing accuracy of depth information extracted from acquired images.

In some embodiments, one or more automatic control feature is not disabled, but accounted for in processing of acquired image data. For example, in some embodiments smartphone control of the imager (e.g., OIS control) is not controlled, but parameter/s used for control of the imager by the smartphone are used to compensate for the imager control (e.g., OIS control). For example, in some embodiments, input/s to a OIS module are used to compensate for (e.g., using image processing of acquired images) hardware movement/s associated with OIS control. Where, in some embodiments, the parameters (e.g., sensor signals e.g., gyroscope and/or accelerometer data for OIS control) used for control of the imager have a higher sample rate than the frame rate (e.g., 100-300 samples per second, or about 200 samples per second, or lower or higher or intermediate ranges or samples per second) than the frame rate of the imager (FPS frames per second e.g., 30-100 FPS e.g., sensor signals are provided at a rate of at least 1.5 times or at least double, or at least triple or lower or higher or intermediate ranges or multiples of the imaging frame rate). The sampled parameters then, in some embodiments, being used in processing of acquired images, for example, to extract depth information e.g., regarding dental features.

Additionally or alternatively to software control of imaging and/or illumination, in some embodiments, control of the smartphone (e.g., camera) is using optical and/or mechanical methods (e.g., alternatively to control using software and/or firmware).

For example, in some embodiments, magnet is used to disable OIS movement of a camera models. The magnet, once positioned behind the CMOS imager, in some embodiments prevents OIS function. In some embodiments, the magnet is a part of (or is hosted by) the add-on. Where positioning and/or magnet type (e.g., size, strength) is customized e.g., per smartphone model. Where customization is in production of the add-on and/or in incorporating of the magnet onto the add-on e.g., where one add-on model in some embodiments, is used for more than one magnet type and/or position e.g., for smartphone models having similar layout but different imager/s.

At 206, in some embodiments, the add-on is attached to the personal device (e.g., smartphone).

In some embodiments, add-on is mechanically attached to smartphone using a case which surrounds the smartphone, at least partially. Where, in some embodiments, the add-on includes a case e.g., has a hollow into which the smartphone is placed to attach the smartphone to the add-on. Where an exemplary embodiment is illustrated, for example, in FIGS. 24A-C.

In some embodiments, add-on is attached mechanically to a face of the smartphone (E.g., to back face opposite a face including the smartphone scree). In some embodiments, the add-on surrounds one or more optical element of the smartphone.

In some embodiments, attachment is sufficiently rigid and/or static to hold smartphone optical element/s and optical pathways of the add-on in alignment.

In some embodiments, a user is provided with feedback as to the quality of attachment of the add-on to the cell phone. Where, in some embodiments, the user is instructed to reposition the add-on.

In some embodiments, for example, where the add-on only transfers imager FOVs e.g., as single imager FOV, aligning includes aligning and attaching the add on to this optical element e.g., only.

At 208, in some embodiments, calibration is performed. For example, in some embodiments, the add-on is calibrated e.g., once it is attached to a smartphone. Alternatively or additionally, the smartphone is calibrated (e.g., prior to attachment of the add-on). Alternatively or additionally, the smartphone is calibrated (e.g., periodically, continuously) during scanning e.g., during image acquisition.

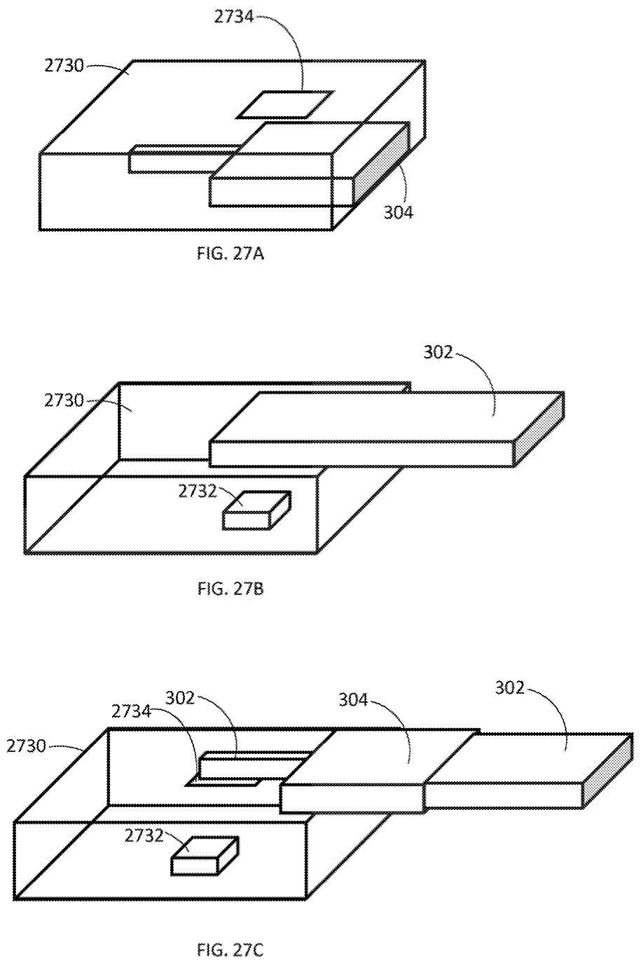

In some embodiments, the add-on attached to the smartphone (and/or the smartphone alone) is calibrated using a calibration element (e.g., calibration jig). For example, after attachment of the add-on to the smartphone and for example, prior to imaging and/or during imaging. In some embodiments, packaging of the add-on includes (or is) a calibration jig. In some embodiments, an add-on is provided as part of a kit which includes one or more calibration element e.g., calibration target and/or calibration jig. Where an exemplary calibration jig is described in FIGS. 27A-C.

In some embodiments, internal feature/s of the add-on are used to calibrate the add-on.

For example, in some embodiments, smartphone camera focus is adjusted for by adjusting software parameter/s of the smartphone by fixing the camera focus e.g., using a high contrast target (e.g., a checkerboard pattern or a face e.g., a simplified face icon). Where, in some embodiments, the calibration target is within the add-on side walls positioned so that target is imaged by the camera without blocking dental images. Where, in some embodiments, the target allows adjustment of camera focus periodically and/or continuously and/or during scanning.

In some embodiments, calibration includes acquiring one or more image, including a known feature, for example, of a known size and/or shape and/or distance away, and/or color. In some embodiments, a known feature includes internal feature/s of the add-on e.g., as appearing in acquired images through the add-on.

For example, in some embodiments, a known color calibration target is used in calibration e.g., of illuminator/s. In an exemplary embodiment, an illuminator (e.g., smartphone flash) is calibrated using image/s acquired of a surface of known color (e.g., white) illuminated by the illuminator. Where, in some embodiments, the images are acquired by an imager which has already been calibrated.

In some embodiments, the calibration is done using the inner part of the periscope that can hold targets for camera focus, resolution measure, color balancing etc. In some embodiments, the inner part of the periscope include an identifier, for example a 2D barcode that is used to identify the specific periscope. This barcode can be used to track the user that is creating the model, can include a security code to reduce the chance of using the wrong periscope (e.g., non-original, e.g., not the right user, e.g., a periscope configured for a different smartphone) with the smartphone application, can be used to track the number of scans a specific periscope was used.

In some embodiments, a calibration target, (e.g., within an inner part of the periscope e.g., of a calibration jig) includes a shade reference that allow the calibration of the specific camera in order to accurately detect the shade of the teeth that are being imaged. The Shade reference, in some embodiments, includes shades of white e.g., as appear in VITA shade guides.

In some embodiments, a known size object when captured in an imager (e.g., by CMOS in pixels) enables an imaged object to pixel conversion. In some embodiments, a known shape enables calibration of tiling (e.g., of the add-on with respect to smartphone optics), for example, by identifying and/or quantifying distortion of a collected image of a known shape.

In some embodiments, calibration includes calibrating (e.g., locking) imager focus and/or exposure. In some embodiments, calibration includes calibrating intrinsic parameter/s of the camera, for example, one or more of; effective focal length, distortion, and image center. In some embodiments, calibration includes calibrating a spatial relation between the add-on and the smartphone camera and/or a spatial relation between at least one pattern projector and at least one camera of the smartphone.

In some embodiments, calibration is performed (e.g., alternatively or additionally to other calibration/s described in this document) during image acquisition using the add-on. For example, in some embodiments, one or more calibration target appear within a FOV of an imager being calibrated during acquisition of images of dental features using the imager. For example, where calibration target/s are disposed on inner surface/s of the add-on. In some embodiments, during processing of acquired images, a CMOS feature of jumping between register value sets, for one or more register (e.g., two) is used. For example, in some embodiments, acquired images have at least two ROIs, one for dental features and one for calibration element/s e.g., within the add-on. Where in some embodiments, focus and/or zoom is changed when switching between the ROIs, evaluating of the two ROIs enabling verification of calibration and/or re-calibration e.g., during scanning.

In some embodiments, calibration information is used as input/s to software for control of smartphone e.g., as described regarding step 204.

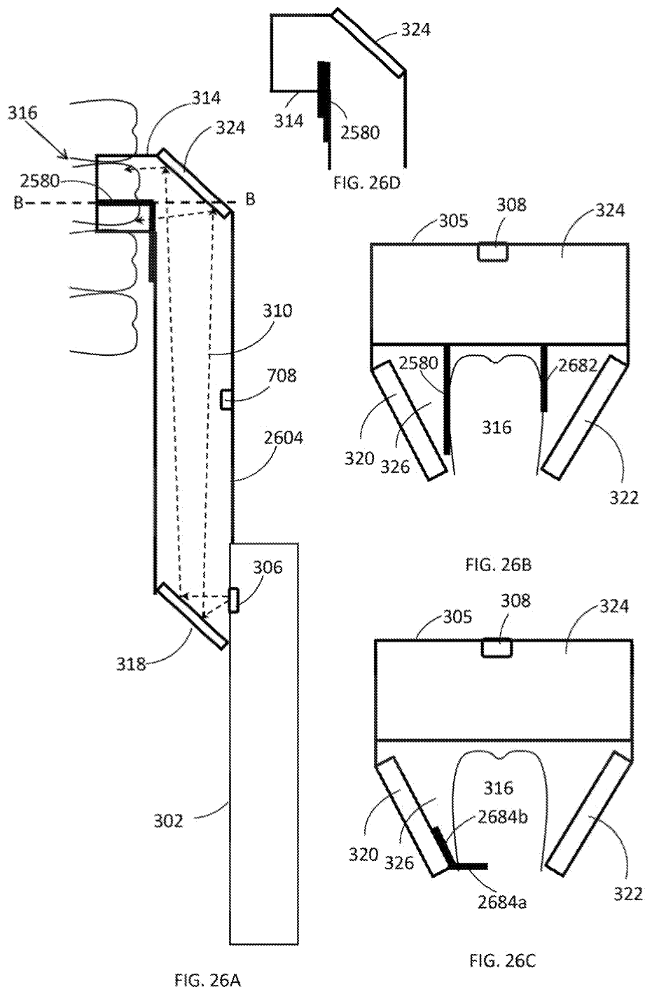

In some embodiments, where the add-on includes a probe (e.g., add-on 2604 FIGS. 26A-C) the probe of the add-on is calibrated e.g., after the add-on is coupled to the smartphone and/or after positioning (e.g., unfolding) of the probe. In some embodiments, calibration includes determining (e.g., by a processor) of a depth position of the probe e.g., probe tip e.g., with respect to the add-on and/or other feature/s. In some embodiments, an image acquired including the probe e.g., without patterned light, is used to determine a position of the probe e.g., with respect to calibration target/s also imaged and/or known imaging parameter/s e.g., focus.

A potential advantage of calibrating position of the probe and/or probe tip e.g., when the probe is in an extended configuration (e.g., unfolded) is more accurate determining of the position of the probe tip. In some embodiments, each time a retractable (e.g., foldable) probe is extended, calibration is performed or, every few extensions e.g., 1-10 extension and retraction cycles. Given, for example, that in some embodiments, mechanics of unfolding of the probe tip positions the probe tip, with respect to the adaptor and/or smartphone results in variation of exact positioning of the probe tip e.g., from unfold to unfold.

In some embodiments, patterned light e.g., produced by a pattern projector, is used in calibration. In some embodiments, image/s acquired under illumination with patterned light are used to configure (e.g., lock) imager focus and/or exposure. In some embodiments, patterned light is used to calibrate intrinsic parameter/s of the camera, for example, one or more of; effective focal length, distortion, and image center. In some embodiments, patterned light is used to calibration a spatial relation between the add-on and the smartphone camera and/or a spatial relation between at least one pattern projector and at least one camera of the smartphone.

At 210, optionally, in some embodiments, one or more fiducial is attached to the subject.

At 212, optionally, one or more mirror is attached to the subject.

In some embodiments, attachment of fiducial/s and/or mirror/s is by positioning a check retractor (e.g., by the user). In some embodiments, a check retractor which does not include fiducial/s and/or mirrors is attached e.g., by the user.

In some embodiments, the subject bites down one or more biter of the cheek retractor. For example, to hold the cheek retractor in a known position with respect to dental feature/s. In some embodiments, one or more back side cheek retractor is positioned. In some embodiments, a cheek retractor and back side cheek retractor are a single connected element.

At 214, in some embodiments, the mouth is scanned using the add-on attached to the smartphone.

In some embodiments, the add-on is inserted into the mouth and moved around within the mouth while collecting images. For example, in some embodiments, a user moves the add-on within the mouth using movement along dental arc/es that are generally used during tooth brushing.

In some embodiments, the user does not view the screen of the smartphone during scanning. Optionally, the user receives aural feedback broadcast by the smartphone during scanning. In some embodiments, the user views the smartphone screen after scanning to receive feedback about the quality of the scan, for example, direction to scan particular areas which were e.g., insufficiently scanned or not scanned.

In some embodiments, the add-on is not inserted into the mouth and images outside surfaces of teeth directly and, in some embodiments, images internal surfaces e.g., lingual surface/s of teeth via reflections onto mirror/s.

In some embodiments, internal mirror/s have fixed position with respect to dental feature/s and/or fiducials.

In some embodiments, scanning includes collected images of dental features illuminated, for example, with patterned optical light.

In some embodiments, illumination is without patterned light (e.g., using ambient illumination and/or non-patterned artificial illumination).

In some embodiments, scanning includes fluorescence measurement/s are collected, by illuminating dental feature/s (e.g., teeth) with UV light and acquiring visible and/or IR light reflected by the features. For example, in some embodiments, incident UV light incident on dental features causes green fluorescence for enamel regions and red fluorescence indicating presence of bacteria. Where, in some embodiments, the add-on includes one or more UV illuminator for projection of UV light onto dental feature/s.

In some embodiments, scanning includes optical tomography, for example, illuminating dental feature/s (e.g., teeth) with visible and/or near infrared light (NIR). With a wavelength of, for example, 700-900 nm, or about 780 nm, or about 850 nm, or lower or higher or intermediate wavelengths or ranges. Where, in some embodiments, the add-on includes one or more NIR LED or LD (laser diode). In some embodiments, scattered visible and/or NIR light images are used to detect caries inside the tooth, for example inside the enamel in the interproximal areas between two teeth.

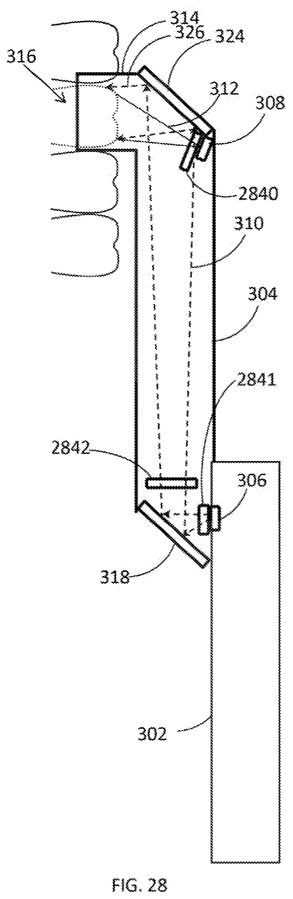

In some embodiments, illumination is using polarized light. For example, according to one or more feature as illustrated in and/or described regarding FIG. 28. Where, in some embodiments, light gathered into one or more imager is polarized, where the polarizing of the gathered light is, in some embodiments, aligned to that used in illumination. Potentially meaning acquired images more accurately include light reflected by dental feature surfaces e.g., as opposed to light absorbed and scattered within dental feature/s before being captured in image/s.

In some embodiments, polarizing of the gathered light is cross-polarized to that of illumination, for example, potentially meaning acquired images include light scattered by dental feature/s before capture in image/s

In some embodiments, the smartphone imager focal distance is adjusted for acquisition of patterned light incident onto dental feature/s. In some embodiments, resolution and/or compression of images acquired is selected to maximize data within images including patterned light.

In some embodiments, during scanning the smartphone imager focus is scanned over a plurality of focus distances, for example, over 2-10, or 2-5, or three different focus distances. For example, where focal distances range from 50-500 nm, where, in some embodiments, three exemplar focal distances are 100 mm, 110 mm, 120 mm. In some embodiments, focal distances are selected based on a distance between the add-on and dental features to be scanned.

Where, in some embodiments, software installed on the smartphone controls the smartphone imager during scanning to provide different focal distances.

In some embodiments, a first imager is used to image outside the mouth e.g., outer surface/s of teeth during scanning inside the mouth e.g., by a second imager or imagers. Where, in some embodiments, the first imager is a smartphone imager directly acquiring images and the second imager FOV is transferred by the add-on. In some embodiments, the first imager is a wide angel imager, and the second imager is a narrow angle imager. In some embodiments, images collected by the first image are used to increase the accuracy of a 3D model of a plurality of teeth in a jaw (e.g., a full jaw). For example, in some embodiments, images collected using the first imager capture larger regions e.g., of external dental features and these images are used to correct accumulated error/s in scanning along a jaw. Where the accumulated errors, in some embodiments, are associated with the narrow FOV of the second imager and/or movement during imaging.

In some embodiments, software downloaded on the smartphone (e.g., at step 204) controls illuminators of the smartphone during scanning. For example, switching illuminators (e.g., LED illuminator/s e.g., via LED chips). Where, in some embodiments, switching is between patterned illumination and un-patterned illumination. Images including patterned light incident on dental features, for example, being used to generate model/s (e.g., 3D model/s) of the dental features and un-patterned light providing color and/or texture measurement of the dental features.

In some embodiments, scanning includes collection of images with a single imager and/or a single FOV. In some embodiments, multiple imagers and/or multiple FOVs are used. Where, in some embodiments, a FOV of a single imager is split into more than one FOV. In some embodiments, imaging is via one or more FOV emanating from an add-on and optionally, in some embodiments, directly via a smartphone imager. Where, FOVs emanating from the add-on include, in some embodiments, smartphone imager FOV transferred through the add-on and/or FOV of imager/s of the add-on.

In some embodiments, multiple images are collected simultaneously e.g., by different imagers. In some embodiments, images from different directions with respect to the add-on and/or smartphone are collected e.g., simultaneously.

At 216, in some embodiments, a user is guided in scanning, for example before during and/or after scanning. e.g., by user interface/s of the smartphone. Where, in some embodiments, guiding includes aural clues. Where, in some embodiments, the user views images displayed on the smartphone directly or via reflection in one or more mirror. Where, in some embodiments, the reflection is in a mirror of the add on. Where, in some embodiments, the reflection is in an external mirror.

In some embodiments, for example, when the smartphone has a screen on his back side, or when, during scanning the smartphone screen is facing the user (e.g., imaging is via an imager on a front face of the smartphone) a user directly views the smartphone screen (or a portion of the screen) during scanning.

At 218, in some embodiments, scanning data is evaluated.

In some embodiments, evaluation of data includes generating model/s of dental features using collected images. For example, 3D models.

In some embodiments, imaged deformation of structured light incident on 3D structures is used to re-construction 3D feature/s of the structures. For example, based on calibration of deformation the structured light.

In some embodiments, for example, where patterned light is not used SFM (structure from motion) technique/s and/or deep learning networks are used to generate 3D model/s from acquired 2D images and optionally the IMU sensor data.

In some embodiments, scan data is evaluated to provide measurement or and/or indicate change/s in one or more of degree of misalignment of the teeth, the shade or color of each tooth surface, how clean is the area between metal orthodontic braces, what is the degree of plaque and/or tartar (dental calculus) is on one or more tooth surface, detecting caries (dental decay, cavities) on and/or inside the teeth and/or their location on a 3D model, detecting tumors and/or malignancies and/or their location on the 3D model.

At 220, in some embodiments, a healthcare professional receives the data evaluation and, in some embodiments, responds to the data evaluation. For example, indicating that the subject should perform an action, for example, book an in-person appointment. For example, changing a treatment plan.

At 222, in some embodiments, communication to the user is performed e.g., via the smartphone. For example, instructions from the healthcare professional. For example, to perform one or more of; aligning the teeth (e.g., use aligners), whiten the teeth, brush between orthodontic braces, brush a specific tooth (e.g., with a lot of plaque), set appointment for tartar (dental calculus) removal, set a dentist, X-ray, or physical test appointment.

### Exemplary Add-On

FIG. 3A is a simplified schematic side view of an add-on 304 connected to a smartphone 302, according to some embodiments.

Description of elements in FIG. 3A, in some embodiments, relates to, but should not be understood as necessarily limiting similarly numbered elements in other Figures of this document.

In some embodiments, add-on 304 includes a housing which holds and/or provides support to optical element/s of the add-on and/or attachment to smartphone 302. Housing e.g., delineated by outer lines of add-on 304.

In some embodiments, add-on 304 includes a slider 314 which local to dental features 316 to be scanned. In some embodiments, slider 314 is disposed at a distal end of add-on 304. In some embodiments, slider 314 is sized and/or shaped to hold dental feature/s 316 and/or to guide movement of the add-on within the mouth the shape of slider 314 with respect to teeth preventing movement in one or more direction. In some embodiments, slider 314 directs and/or includes optical element/s to direct optical path/s (e.g., of imager/s and/or lighting) to and/or from the dental feature/s 316.

In some embodiments, add-on 304 provides an optical path for one or more imager FOV 310 e.g., as illustrated in FIG. 3A by dashed arrows. In some embodiments, add-on 304 provides an optical path for light 312 from one or more illuminator and/or projector 308 e.g., as illustrated in FIG. 3A by solid arrows. Where, in some embodiments, projector 308 projects patterned light.

In some embodiments, the optical path is provided by one or more mirror 318, 324.

### Exemplary Multi-View Capture

FIG. 3B is a simplified schematic sectional view of an add-on, according to some embodiments.

FIG. 3C is a simplified schematic sectional view of an add-on, according to some embodiments.

In some embodiments, FIG. 3B and FIG. 3C illustrate a cross sectional view of add-on 304 of FIG. 3A, e.g., taken along line AA, e.g., showing a sectional view of slider 314.

FIG. 3B, in some embodiments, illustrates FOV 310 of imager 306 which, in some embodiments, is split by mirrors 320, 322.

In some embodiments, the add-on includes both of mirrors 320, 322, e.g., providing views (e.g., to imager 306) of both lingual and buccal sides of dental feature/s 316. In some embodiments, however, the add-on includes one of mirrors 320, 322, the add-on, for example, providing views (e.g., to imager 306) of occlusal and one of lingual and buccal sides of dental feature/s 316.

In some embodiments FOV 310 of imager 306 is illustrated using dashed line arrows, both in FIG. 3A and FIG. 3B.

FIG. 3C, in some embodiments, illustrates FOV 312 of projector 308, which, in some embodiments, is split to be directed to the sides of tooth 315.

In some embodiments FOV 312 of projector 308 is illustrated using solid arrows, both in FIG. 3A and FIG. 3C.

In some embodiments, pattern projector 308 is located on a top side of periscope 304 e.g., a top side of housing 305. In some embodiment, projected light (e.g., patterned light) illuminates an occlusal part of tooth 316 and/or two sides of. tooth 316 e.g., the buccal and lingual sides through mirrors 322 and 320.

In some embodiments, view/s of the dental feature/s 316 illuminated by patterned light 312 are reflected back towards imager 306 by mirrors 324, 318. Where side view/s of dental feature 316 (e.g., buccal and lingual views e.g., when the dental feature is a molar) are reflected by mirrors 320, 322 to mirrors 324, 318.

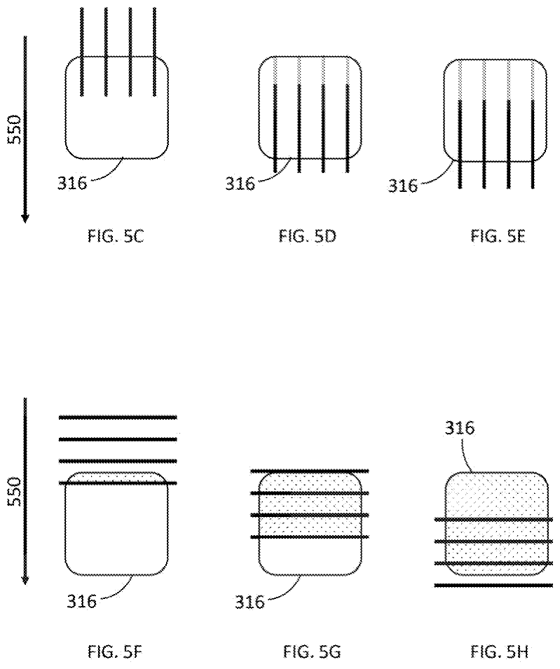

In some embodiments, light reflected back to imager 306 includes 3 FOVs combined together, e.g., as illustrated in FIG. 5A and/or FIG. 5B.

Optionally, in some embodiments, periscope 304 includes (e.g., in addition to a pattern projector) a non-patterned light source, e.g., a white LED, potentially enabling acquisition of colored image/s of dental feature/s. In some embodiments, one or more of mirrors 320, 322, 324 are heated potentially reducing condensation e.g., condensation associated with the subject's breath inside the mouth while scanning. In some embodiments, heating of the mirrors is provided by one or more heater PCB attached to the back side of the mirror and/or mirrors. In some embodiments, heat is transferred from illuminator/s to the mirrors. In some embodiments heat is transferred from the smartphone body and/or electrical parts of the add-on and/or smartphone. Where transfer of heat is by using a metal element (e.g., solid metal element) and/or metal foil and/or heat pipe/s. In some embodiments, the mirrors include aluminum (e.g., for good heat transfer). In some embodiments, one or more of the mirrors have an anti-fog and/or other hydrophobic coating potentially preventing and/or reducing fog on the mirror and/or mirrors. In some embodiments, the adjacent teeth (e.g., to a tooth local to slider 314) and/or other teeth in the jaw are captured using another camera and/or imager of the smartphone. In some embodiments, the smartphone captures image/s in parallel (e.g., simultaneously and/or without moving the smartphone and/or add-on) using two different cameras. In some embodiments, image/s from the first camera is used to capture the teeth from 3 directions e.g., as illustrated in FIGS. 3A-C. Where, in some embodiments, image/s from the second camera capture more teeth along the dental arch. In some embodiments, the second image/s are used to reduce the accumulated error e.g., as described elsewhere in this document. In some embodiments, the second camera is located on an opposite side of the smartphone, for example a “selfie” camera, and mirrors, in some embodiments, are used to direct the FOV of the second camera to capture large parts, e.g., more than 2 teeth, e.g., at least quarter, half, three quarters of the dental arch, while the first scanner is scanning, for example, an individual tooth. In some embodiments, the second camera captures the opposite dental arch to the first camera.

In some embodiments, the measurement system (e.g., including an add-on) includes multiple pattern projectors and/or illuminators. In some embodiments, for example, there are three different pattern projectors, e.g., one for each of lingual, buccal and occlusal sides of dental features.

In some embodiments, where the pattern projector and imager are located in different positions in one or more direction, pattern projected is configured so that lines of a projected pattern remain, e.g., for each split of the FOV of the imager, at an angle (e.g., as quantified elsewhere in this document) to a direction of scanning.

In some embodiments, multiple pattern projectors are located such that the difference between the optical axis of imaging FOVs and projected FOV is large enough to produce depth by analyzing the obtained images of the projected pattern with the imagers.

In some embodiments, there are two different pattern projectors that are placed at about 45 degrees with respect to a tooth, and 90 degrees between each other, and allow each projector to capture occlusal and lingual surfaces of the tooth using one projector and the occlusal and buccal using another projector. Or the occlusal and buccal surfaces using one projector and the occlusal and lingual using another projector. In some embodiments, the projectors are controlled to allow one of the projector at a time to transmit light (or to transmit patterned light) potentially preventing patterns from both projectors being incident on the same area (e.g., occlusal surface). Where two patterns incident on a same surface potentially reduces accuracy of depth calculation from acquired image/s of the surface. In some embodiments, camera exposure time is synchronized with selection of projection potentially producing acquired images which include a single pattern from a single projector.

In some embodiments, FOV splitting, for example, as illustrated in and/or described regarding FIG. 3B, (e.g., by mirrors 320, 322) is performed closer to imager 306, for example between smartphone 302 and mirror 318. Splitting imager 306 FOV in this space, potentially involving splitting element/s e.g., where FOV 310 expands in extent moving in a direction away from camera 306. In some embodiments, mirrors having a size similar to a lens diameter of smartphone camera 306, for example mirror/s having reflecting surfaces, in one or more dimension of 0.5-10 mm, or 0.1-5 mm, or 0.1-3 mm, lower or higher or intermediate ranges or dimensions. Distal to this splitting element, in some embodiments, the add-on consist of two periscopes each having 2 mirrors, performing the function of mirror 318 and mirror 324 in FIG. 3A and FIG. 3B, to transfer the split FOVs to the dental feature/s 316. Where the add-on does not include mirrors 322 and 320 potentially enabling a smaller add-on.

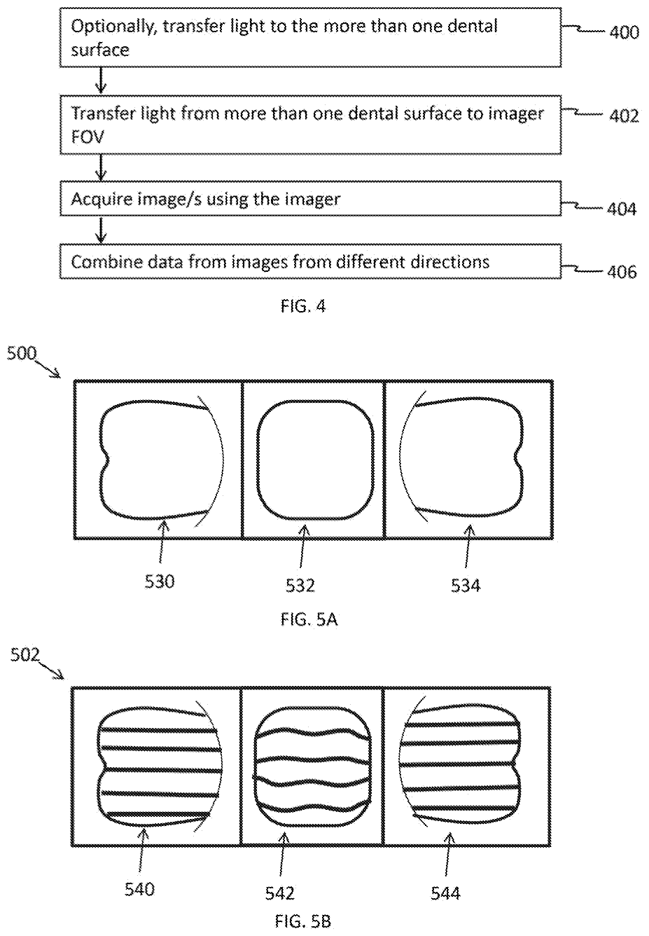

FIG. 4 is flowchart of a method of oral measurement, according to some embodiments.

At 400, in some embodiments, optionally, light is transferred to the more than one dental surface e.g., more than one of occlusal, lingual, and buccal surfaces of one or more tooth (and/or dental feature e.g., dental prosthetic). Where, in some embodiments, light is patterned light. Where transfer, in some embodiments, is via one or more optical element e.g., mirror and/or lens.

Where, in some embodiments, light from a single light source is split into more than one direction to illuminate more than one surface of a dental feature (e.g., tooth)

At 402, in some embodiments, light from more than one dental surface is transferred to an imager FOV (or more than one imager FOV). Where transfer is via one or more optical element. Where, in some embodiments, a single imager FOV is split into more than one direction e.g., by mirrors, the FOV being directed towards more than one surface of a dental feature (e.g., tooth).

At 404, in some embodiments, image/s are acquired using the imager/s.

At 406, in some embodiments, images acquired are processed, for example, where images are stitched combined e.g., in generation of a model e.g., 2D model of the feature/s (e.g., dental feature/s) imaged. In some embodiments, the images are combined using overlapping region/s between images. For example, where a top view of the tooth e.g., as seen in central panel of FIG. 5A and FIG. 5B has an overlapping region e.g., with each of the side views on the side panels of the figures.

FIG. 5A is a simplified schematic of an image 500 acquired, according to some embodiments. Image 500, in some embodiments, shows tooth 316 image when tooth 316 is illuminated with non-patterned light. Image 500, in some embodiments, showing occlusal 532, lingual 530, and buccal 534 views of tooth 316. Where, in some embodiments, image 500 is a single image captured with an imager, where the FOV of the imager has been split e.g., as described regarding FIG. 3B and/or elsewhere in this document.

### Exemplary Illumination, Exemplary Light Patterning

FIG. 5B is a simplified schematic of an image 502 acquired, according to some embodiments.

Image 502, in some embodiments, shows tooth 316 when illuminated with patterned light e.g., by a pattern projector e.g., pattern projector 308. In some embodiments, pattern projector 308 includes a single optical component providing optical power (e.g., to focus the light) and a pattern e.g., the element including one or more feature as illustrated in and/or described regarding FIG. 20A and/or FIG. 20B.

In some embodiments, the projected pattern (e.g., used to determine the depth information) includes straight lines e.g., parallel lines.

Where, in some embodiments, image 502 is a single image captured with an imager, where the FOV of the imager has been split e.g., as described regarding FIG. 3B and/or elsewhere in this document. Where, in some embodiments, image 502 is captured using illumination from a single pattern projector, where light of the pattern projector has been split e.g., as described regarding FIG. 3C and/or elsewhere in this document.

FIGS. 5C-E are simplified schematics of patterned illumination with respect to a dental feature, during scanning, according to some implementations.

FIGS. 5F-H are simplified schematics of patterned illumination with respect to a dental feature, during scanning, according to some embodiments.

In some embodiments, arrow 550 indicates a scanning direction, with respect to dental feature 316.

FIGS. 5C-E illustrate an embodiment where a scan pattern (indicated by black lines) is parallel to scanning direction 550. Where the figures show, in some embodiments, movement of the patterned light during scanning.

Grey lines in FIGS. 5D-E illustrate regions of dental feature 316 for which the patterned light provides depth information.

FIGS. 5F-G illustrate an embodiment where a scan pattern (indicated by black lines) is perpendicular to scanning direction 550. Where the figures show, in some embodiments, movement of the patterned light during scanning. Where dot-shaded portions of dental feature 316 indicate regions of dental feature 316 for which the patterned light provides depth information.

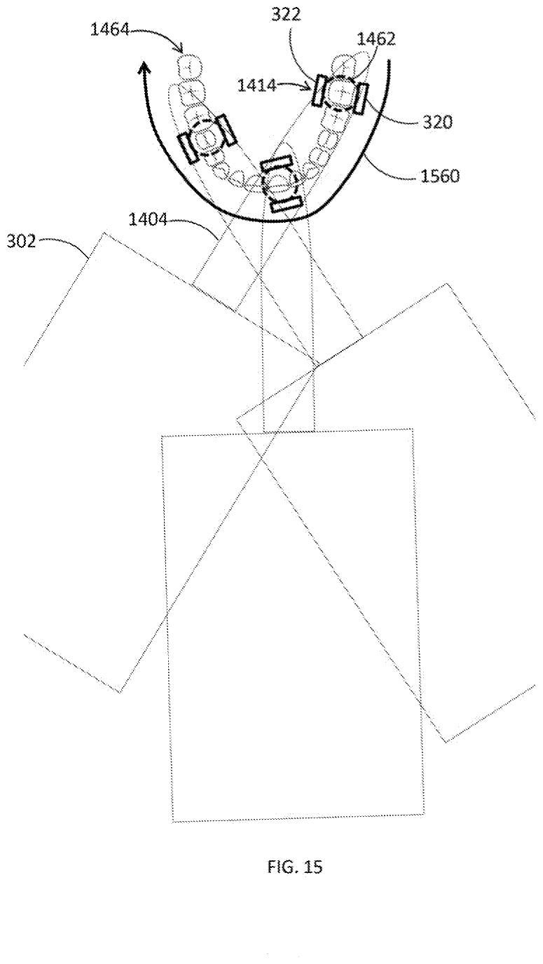

In some embodiments, a direction of straight line pattern projected light is perpendicular (or about perpendicular), or at least 20 degrees, or at least 30 degrees, or at least 45 degrees to scanning direction 550. Where, in some embodiments, scanning movement is along a dental arch (e.g., as illustrated in by arrow 1560 FIG. 15).

In some embodiments, projected lines are monochrome e.g., including one color of light e.g., white light. In some embodiments, projected lines are colored e.g., having different colors. In some embodiments, the pattern projector projects a single pattern, (potentially reducing complexity and/or cost of the pattern projector). In some embodiments, the pattern projector projects a set of patterns.

In some embodiments colored light includes red, green and blue light and/or combinations thereof. In some embodiments colored light includes at least one white line. Potentially such colored light and optionally white light enabling collection of color information regarding dental features and/or real color reconstruction of scanned dental features (i.e., teeth and gingiva).

FIG. 28 is a simplified schematic side view of an add-on 2804 connected to a smartphone 302, according to some embodiments.

For example, including one or more feature as described regarding use of polarization in step 214 FIG. 2B, in some embodiments, add-on includes one or more polarizing filter 2840, 2841 and/or one or more polarized light source 308.

Where, in some embodiments, add-on 2804 includes a polarized light source e.g., a polarized pattern projector 308 (and polarizer 2840, in some embodiments, is absent). For example, where the polarized light source includes e.g., laser diode/s, VCSEL/s (vertical cavity surface emitting laser).

In some embodiments, polarized light is projected from projector 308 (optionally passing through polarizer 2840) to illuminates dental feature/s 316. A portion of incident light on dental features, which is mainly polarized, is back reflected from surfaces of dental feature/s. A portion of the light is scattered within the teeth and/or soft tissue becoming un-polarized.

In some embodiments, the optical path of add-on 304 includes a second polarizer (e.g., one of polarizers 2841, 2842) which polarizes light received. Depending on the direction of polarization of the polarizer (2841 or 2842) reflected or scattered light is received by imager 306.

In some embodiments, polarizers 2840, 2841, 2842 are linear polarizers, where the polarization direction is parallel.

In some embodiments, a polarization direction of polarizers 2840 and 2842 is parallel to the image plane of FIG. 28. In this case, a proportion of the light received by imager 306 which is light previously scattered at dental features is reduced, potentially resulting in improved contrast of acquired images of the dental surfaces.

In some embodiments, polarizers 2840, 2841, 2842 are crossed, e.g., perpendicular (or about perpendicular). For example, in some embodiments, a polarization direction of polarizer 2840 is parallel to the image plane of FIG. 28 and the polarization direction of polarizers 2841, 2842 is perpendicular to the image plane. Potentially, specular reflection from the dental surfaces incident on imager 306 is reduced the image mainly being formed of scattered light of the projected pattern.

Potentially, in some embodiments, images acquired using aligned polarization have improved contrast e.g., of patterned light incident on dental surface/s.

In some embodiments, images acquired using cross polarizers are used to provide information regarding demineralization of enamel e.g., potentially providing early indication of onset of caries. For example, using and/or including one or more feature described in one or both of:
“Monitoring tooth demineralization using a cross polarization optical coherence tomographic system with an integrated MEMS scanner” by Daniel Fried et al, Proc SPIE Int Soc Opt Eng. 2012 Feb. 9; 8208: 82080I, which reference is herein incorporated by reference in its entirety.
“Evaluation of cross-polarized near infrared hyperspectral imaging for early detection of dental caries” by Peter Usenik et al, February 2012 Proceedings of SPIE—The International Society for Optical Engineering 8208:10-DOI:10.1117/12.908763, which reference is herein incorporated by reference in its entirety.

In some embodiments, the add-on includes a projector having an illuminator and a patterning element but lacking a projection lens, potentially reducing cost of the projector potentially enabling an affordable single use add-on.

In some embodiments, a pattern and/or projection lens is directly connected to the smartphone (e.g., by a sticker and/or using temporary adhesive) to the smartphone case and/or outer body and/or to a smartphone cameras array glass cover. In some embodiments, the adhered element alone is an add-on to the smartphone. In some embodiments, the directly connected element (e.g., sticker) is used for dental scanning (e.g., with an add-on), and is then removed. In some embodiments, the sticker is a single-use sticker, for example, being discarded after scanning.

In some embodiments, a pattern projector illuminates with parallel lines of light. In some embodiments, axes of the lines are orientated perpendicular to a base line connecting the imager and the projector.

In some embodiments depth is calculated from the movement of the lines across their short axes. Where the optical path of the patterned light has been changed e.g., by mirrors, the same technique is used, however the baseline is determined between the projector and the camera mirror virtual positions.

In some embodiments, if the baseline is parallel to the orientation of the pattern lines it is not possible to determine depth information from acquired images.

In some embodiments, the pattern projector projects lines and the add-on and/or projector are configured so that long axes of lines are perpendicular to a line connecting the camera and the projector. Depth variations then, in some embodiments, move the pattern lines perpendicular to the direction of the base line. In some embodiments, during estimation of line movements, depth is estimated as well. Where mirror splitting of projected patterned light is employed, the base line is found between the projector and camera virtual positions (the positions that would create the same pattern/image if there were no mirrors). In some embodiments, other pattern/s are projected e.g., a pseudo random dots pattern where, in some embodiments, the depth is determined for any orientation of the base line.

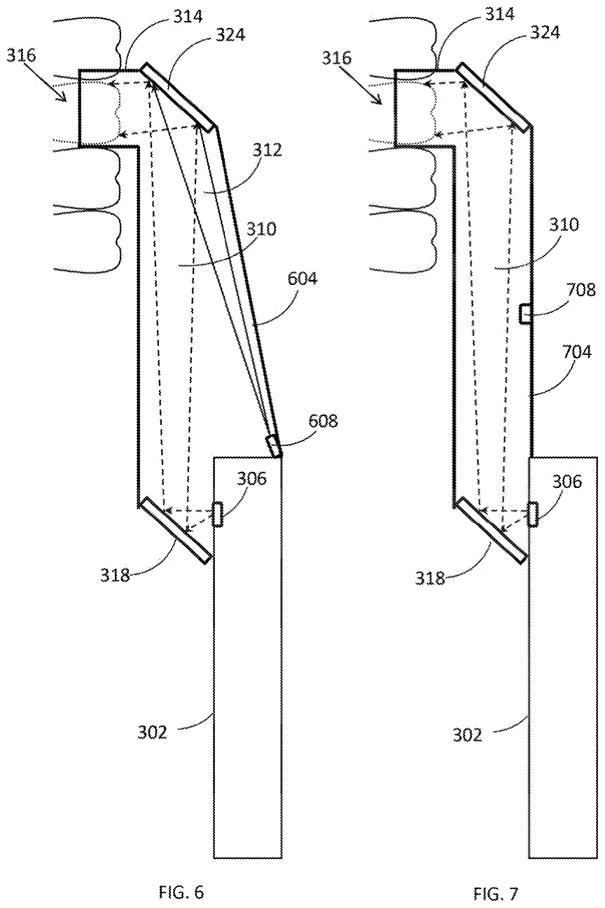

FIG. 6 is a simplified schematic side view of an add-on 604 connected to a smartphone 302, according to some embodiments.

In some embodiments, add-on 604 includes one or more feature of add-on 304 FIG. 3A and/or FIG. 3B.

For example, in some embodiments, an illuminator 608 projects light through mirror 324 onto an occlusal part of tooth 316. In some embodiments, pattern projector light is transferred by mirror 324 and mirrors 320 and 322 to the buccal and lingual sides of tooth 316 e.g., as illustrated at FIG. 3A and/or FIG. 3B and/or FIG. 3C.

In some embodiments, light for an illuminator 608 (which in some embodiments is a pattern projector) is supplied by smartphone 302. For example, by a smartphone LED 608. In some embodiments, the light is projected through one or more optical element (e.g., lens and/or pattern element) where, in some embodiments, add-on 604 hosts the optical element/s.

### Additional Exemplary Add-ons

FIG. 7 is a simplified schematic side view of an add-on 704 connected to a smartphone 302, according to some embodiments.

In some embodiments, add-on 704 includes one or more feature of add-on 304 FIG. 3A and/or FIG. 3B.

Optionally, (e.g., additionally or alternatively to a pattern projector) in some embodiments, add-on 704 includes an illuminator 708. Where, in some embodiments, illuminator 708 supplies non-structured light. In some embodiments, illuminator 708 provides white (e.g., uniform) illumination potentially enabling acquisition of “real color” image/s. In some embodiments, a non-structured light illuminator is lit alternatively with a pattern projector, dental feature/s being alternatively illuminated with structured and non-structured light.

In some embodiments, when dental features are only illuminated using only patterned light, real color images are reconstructed using patterned light images. Potentially reducing complexity and/or cost of the system and/or add-on.

FIG. 8A is a simplified schematic top view of a portion of an add-on, according to some embodiments.

FIG. 8A illustrates a top view of mirrors 322, 320 and 324 with respect to tooth 316.

In some embodiments one or both of mirrors 322 and 320 has a tilt in the horizontal direction e.g., with respect to a central long axis of the add-on and/or smart phone e.g., as illustrated in FIG. 8A.

In some embodiments, e.g., as shown in FIG. 8A, imaging of buccal and lingual sides of tooth 316 is directly through mirrors 322 and 320 e.g., without passing through mirror 324.

FIG. 8B is a simplified schematic cross sectional view of an add-on, according to some embodiments.

FIG. 8B, in some embodiments, is a cross sectional view of the add-on of FIG. 8A.

FIG. 8C is a simplified schematic cross sectional view of an add-on, according to some embodiments.

FIG. 8D is a simplified schematic cross sectional view of an add-on, according to some embodiments.

FIG. 8E is a simplified schematic cross sectional view of an add-on, according to some embodiments.

In some embodiments, mirrors are cut in a non-rectangular shape, for instance as shown in FIG. 8C e.g., potentially optimizing illumination and/or imaging at overlapping areas of the FOV.

In some embodiments, e.g., as shown in FIG. 8D, the add-on includes only two mirrors at a distal end of the add-on. For example, to scan two sides of the teeth (i.e., lingual+occlusal, or buccal+occlusal). In some embodiments, this configuration uses 2 scans or swipes over the arch to scan it, but still provides mechanical guidance e.g., to assist self-scanning. In some embodiments, data from multiple (e.g., at least 2) swipes is stitched together using common portion/s e.g., using the occlusal side which is common.

In some embodiments, e.g., as shown in FIG. 8E, only 2 mirrors are used. Where, in some embodiments, two mirrors are angled to scan sides of the teeth 816 at about 45 degrees (i.e., one mirror scans lingual+occlusal, and one mirror scans buccal+occlusal). In some embodiments, this configuration uses at least one scan or swipe over the arch to scan it and provides some mechanical guidance.

In some embodiments, as shown at FIGS. 8A-8E imaging is done directly through side mirrors (e.g., 320 and 322) and not through distal mirror 312 as for example in FIG. 3A. In those case the imaging can be done through back mirror 318 in case of folded configuration as shown in FIG. 3A or without it in case mirror 318 is not used.

Similarly, the patterned light is projected directly through said side mirrors (e.g., 320 and 322). For instance, if the pattern projector is located on the top side of the periscope, such as 308 in FIG. 3A, it potentially enables good depth reconstruction for the multiple directions (e.g., 2 or 3 directions shown in FIGS. 8A-8E).

In some embodiments of using a single projector of lines pattern as described before to illuminate the 3 mirrors in FIGS. 8A-8C the side mirrors may be slightly tilted also on the horizontal axis to create an angle of at least 20 degrees between the pattern lines on the tooth buccal and lingual sides and the scanning direction, as described in FIGS. 5A-H.

In some embodiments of using a single projector of lines pattern as described before to illuminate the 2 mirrors in FIGS. 8D-8E the side mirror may be slightly tilted also on the horizontal axis to create an angle of at least 20 degrees between the pattern lines on the tooth buccal and lingual sides and the scanning direction, as described in FIGS. 5A-H.

In some embodiments, for example, where patterned light is projected through the side mirrors, as described in FIGS. 8A-8E, the pattern projector is located having a position within and/or with respect to the add-on body, in at least one direction e.g., in a direction perpendicular to a direction of elongation of the add-on and/or smartphone which is similar (e.g., within 1 cm, or within 5 mm, or within 1 mm) to that of the imager, for instance if the pattern projector is located on the top side of the periscope, such as described in FIG. 3A, it potentially enables good depth reconstruction for multiple directions (e.g., 2 or 3 directions).

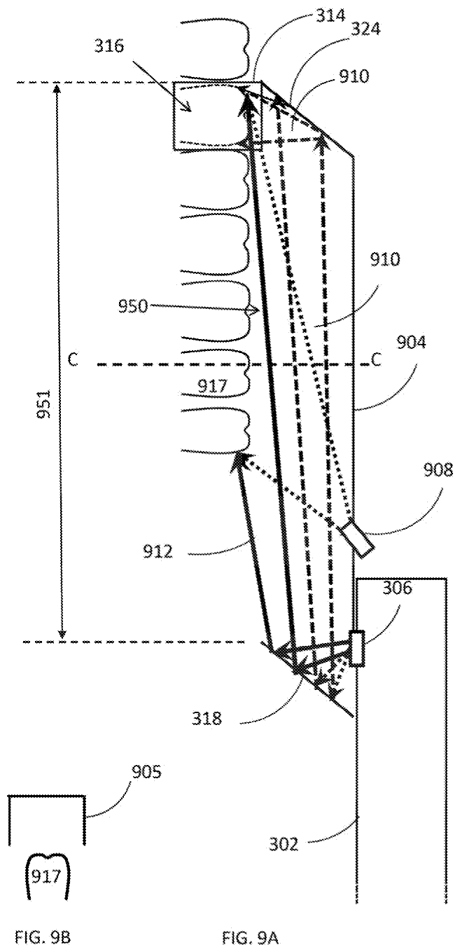

FIG. 9A is a simplified schematic side view of an add-on 904 connected to a smartphone 302, according to some embodiments.

FIG. 9B is a simplified schematic cross sectional view of an add-on, according to some embodiments.

In some embodiments, FIG. 9B illustrates a cross section of add-on 904 of FIG. 9A taken along line CC. For example, showing a relationship between a body 905 of add-on 904 with respect to a dental feature 917.

In some embodiments, the bottom side of the periscope 904 has a wide opening (and/or transparent part), for example, the opening (and/or transparent part) being at least 1-10 cm or at least 1-4 cm, or lower or higher or intermediate widths or ranges, in at least one direction e.g., width 951 is 1-10 cm, or 2-10 cm, or lower or higher or intermediate widths or ranges).

Where the lower opening (or transparent portion) is configured (as shown at FIG. 9B) to enable acquiring images of a “wide range view” (e.g., including a plurality of teeth) in a FOV 912 of imager 306 through add-on 904 e.g., whilst slider 314 guides scanning movement. Where a wide range FOV is illustrated in FIG. 9A by solid arrows.

In some embodiments, narrow range view images are acquire using the add-on, for example as described regarding FIGS. 3A-C. Where a narrow range FOV is illustrated in FIG. 9A by heavy dashed lines.

Where, in some embodiments, add-on 904 acquires images of both wide FOV and small FOV using imager 306. For example, by selecting portions of the imager FOV and/or where imager 306 includes more than one camera e.g., of the smartphone.

In some embodiments, pattern projector 908 illuminates the wide view with patterned light the FOV of pattern projector e.g., as illustrated by dotted line arrows in FIG. 9A.

Alternatively or additionally to pattern projector 908, in some embodiments, add-on 904 includes a pattern projector (not illustrated in FIG. 9A) which illuminates the narrow range FOV with structured light, e.g., as illustrated and/or described regarding projector 308 FIG. 3A and/or FIG. 3C. Where, for example, wide range views are not illuminated (or are mainly not illuminated by patterned light).

In some embodiments, a 3D model is obtained by stitching (combining) of images having smaller FOV e.g., as shown for example at FIGS. 5A-5B with at least one image of a larger FOV 912 e.g., potentially reducing accumulated error/s of stitching. Where, in some embodiments, FOV 912 is at least 10%, or at least 50% or at least double, or triple, or 1.5-10 times a size, in one or more dimension, of the FOV 910 used to generate the 3D model.

In some embodiments, add-on 904 enables acquiring images of a plurality of teeth. Where, an optical path of add-on transfers light of an illuminator 908 (which is in some embodiments a pattern projector) and/or FOV/s 910, 912 of imager 306 through add-on 904 to a wider extent of dental features e.g., whilst slider 314 mechanically guides scanning movement. Where, in some embodiments, the extent is 1-3 cm, in at least one direction, or lower or higher or intermediate ranges or extents.

In some embodiments, a bottom side 950 of periscope 904 is open and/or is transparent. For example, enabling FOV 912 of imager 306 to encompass a wider range of dental features e.g., adjacent teeth to tooth 316 and/or or a full quadrant e.g., as shown in FIG. 9A. In some embodiments, one or both sides (e.g., portions of a body of add-on 904 parallel to a plane of the image of FIG. 9A) of periscope are open and/or include transparent portion/s. For example, potentially widening access of FOV to dental feature/s.

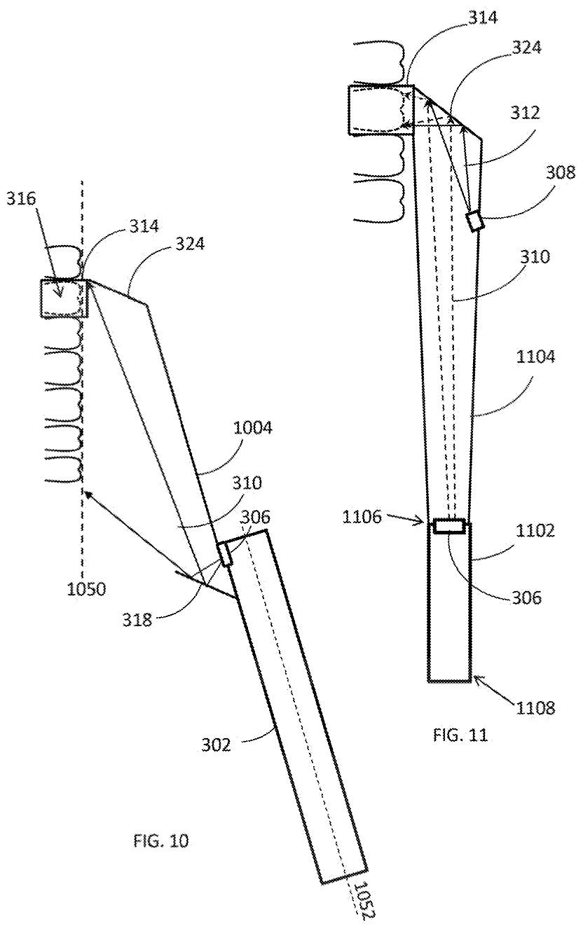

FIG. 10 is a simplified schematic side view of an add-on connected to a smartphone, according to some embodiments.

In some embodiments, e.g., where add-on 1004 has an open bottom side, the intraoral scanner, scans at larger angles to a surface of dental features (e.g., occlusal surface of dental features 316) and/or distances from the surface. Where the angle is, for example, where a central long axis 1052 of smartphone 302 is at an angle of 20-50 degrees or lower or higher or intermediate angles or ranges to an occlusal surface 1050 plane.

Potentially, such angles provide image capture of 5-15, or 11-15 teeth e.g., at a better viewing angle e.g., with more detail, as it is imaged over a larger extent of the camera FOV.

In some embodiments, add-on 1004 includes an illuminator (e.g., pattern projector) and/or is configured to transfer light of such an element. The angle, potentially increasing quality of a projected pattern.

FIG. 11 is a simplified schematic side view of an add-on 1104 connected to an optical device 1102, according to some embodiments.

Where, in some embodiments, optical device 1102 includes an imager 306. In some embodiments, optical device is an intraoral scanner (IOS) and/or an elongate optical device where an FOV 310 of imager 306 emanates from a distal end 1106 of optical device a housing 1102. Where, in some embodiments, housing 1102 is elongate and/or thin (e.g., less than 3 cm, or less than 4 cm, or lower or higher or intermediate dimensions in one or more cross section taken in a direction from distal end 1106 towards a proximal end 1108 of housing 1102).

Where, in some embodiments, add-on 1104 includes mirror 324, in some embodiments, mirrors 320, 322, referring to FIG. 3B and FIG. 3C which in some embodiments, are cross sections of add-on 1104). In some embodiments, add-on 1104 includes a slider 314 e.g., including feature/s as described elsewhere in this document. In some embodiments, add-on includes a pattern projector 308 e.g., including feature/s as described elsewhere in this document.

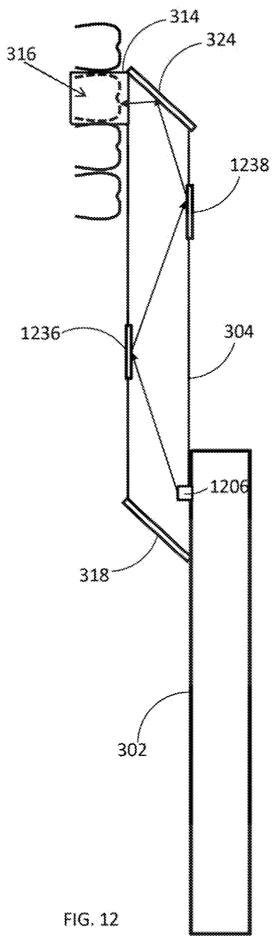

FIG. 12 is a simplified schematic side view of an add-on 304 connected to a smartphone, according to some embodiments.

In some embodiments, add-on 304 includes more than one, or more than two optical elements, or 2-10 optical elements, or lower or higher or intermediate numbers of optical elements for transferring light along a length of the body of the add-on. For example, one or more mirrors 1236, 1238 e.g., in addition to mirrors 318, 324.

Where, in some embodiments, the light is light emanating from a smartphone 302 illuminator 1206 which is transferred through add-on 304 to illuminate dental feature 316. Where, alternatively or additionally, in some embodiments, light is light reflected by dental surface/s which is transferred through add-on 304 to an imager 1206 of add-on 306. Where element 306 of FIG. 12 includes a smartphone illuminator and/or a smartphone imager.

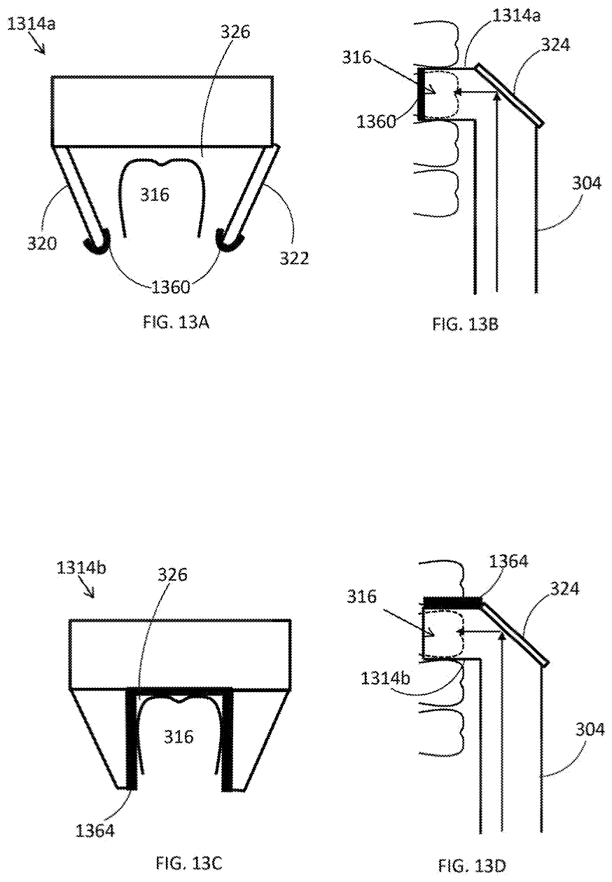

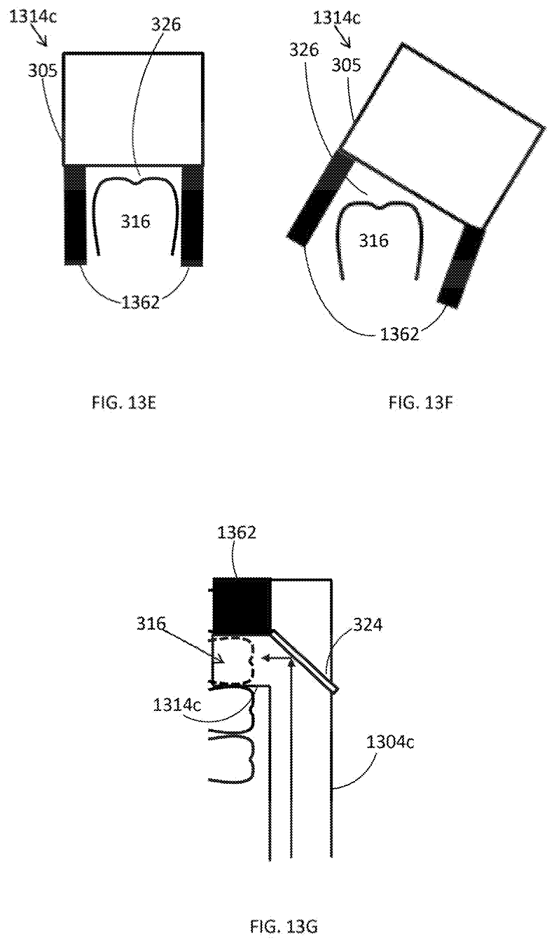

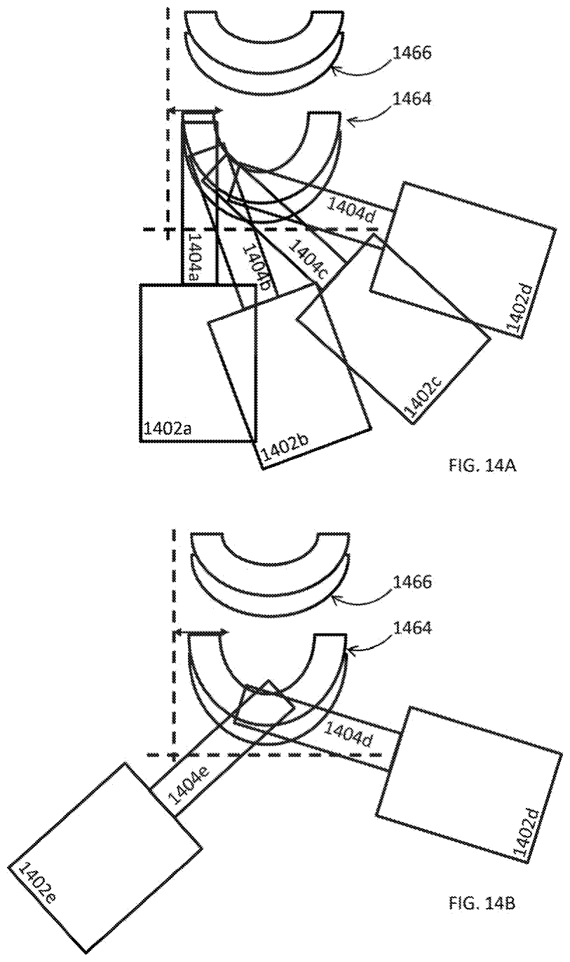

FIG. 13A is a simplified schematic cross sectional view of a slider 1314a of an add-on 1310, according to some embodiments.

FIG. 13B is a simplified schematic side view of a slider 1314a, according to some embodiments.

In some embodiments, FIG. 13B illustrates the slider 1314a of FIG. 13A.

In some embodiments, potentially increasing suitability for self-intraoral scanning, sharp edge/s e.g., edges potentially in contact with mouth soft tissue during scanning, are rounded and/or covered with a soft material 1360. A potential benefit of soft and/or rounded surface/s is improvement of the user experience and/or feeling in the mouth.

In some embodiments, the soft covering includes silicone and/or rubber. In some embodiments, the soft covering includes biocompatible material.

FIG. 13C is a simplified schematic cross sectional view of a slider 1314b of an add-on, according to some embodiments.

FIG. 13D is a simplified schematic side view of a slider 1314b, according to some embodiments.

In some embodiments, FIG. 13D illustrates the slider 1314b of FIG. 13C.

In some embodiments, slider 1314b includes a soft and/or flexible portion 1364 which is deflectable and/or deformable by contact with dental feature/s 316. In some embodiments, flexible portion 1364 includes a ribbon of material on one or more side of an inlet 326 of the slider. Potentially, portion 1364 holds dental feature/s 316 in position e.g., with respect to optical feature/s of the slider. For example, potentially guiding a user in positioning of the add-on with respect to dental feature/s 316.

FIG. 13E is a simplified schematic cross sectional view of a slider 1314c of an add-on, according to some embodiments.

FIG. 13F is a simplified schematic cross sectional view of a slider 1314c of an add-on, according to some embodiments.

FIG. 13G is a simplified schematic side view of an add-on 1304c, according to some embodiments

In some embodiments, FIGS. 13E-G illustrate the same slider 1314c.

In some embodiments, a soft and/or flexible and/or deflectable material “skirt” 1362 is connected to a body 305 of the add-on. Where deflection of the skirt is, for example, illustrated in FIG. 13F. Where skirt 1362 includes, for example, silicone and/or rubber and/or other biocompatible material. In some embodiments, skirt 1362 forms a scanning guide, which in some embodiments, guides the add-on (e.g., during self-scanning) to be centered over the dental arch. In some embodiments, skirt 1362 also retract/s and/or obscures the tongue and/or check potentially reducing interference of these tissue/s to acquisition of images of dental feature/s.

### Exemplary Movement of Add-On During Scanning, Exemplary Slider Rotation

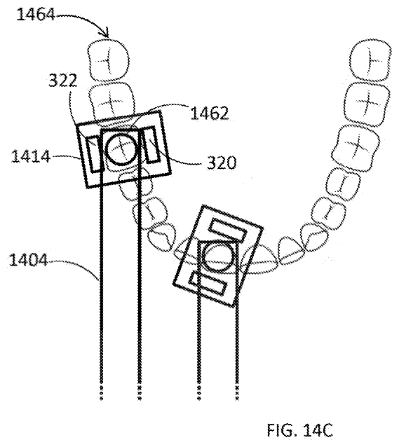

FIG. 14A-B are simplified schematics illustrating scanning a jaw with an add-on, according to some embodiments.

Where, in some embodiments, the add-on includes a distal portion 1404a, 1404b, 1404c, 1404d, 1404e extending away from a body where, in some embodiments body attaches the add-on to a smartphone. For simplicity, in illustrations FIG. 14A-B the smartphone body and smartphone are illustrated as a single component 1402a, 1402b, 1402c, 1402d, 1402e.

FIG. 14A, in some embodiments, illustrates scanning of a portion of lower jaw 1464, for example, a half of jaw 1464, starting at a most distal molar where a distal end of distal portion 1404a is aligned over the distal molar, and extending to a region of jaw 1464 including incisors, where the distal end of distal portion 1404d is aligned over the region.

In some embodiments, for example, to enable accessing of the second portion of the jaw, an orientation of the distal portion of the add-on is changed (e.g., as well as an orientation of a body portion of the add-on and/or an orientation of the smartphone). For example, as illustrated in FIG. 14B by change in orientation e.g., with respect to jaw 1464, between portions 1404d, 1402d to that of portions 1404e, 1404e. In some embodiments, a change in orientation is required to maintain alignment of the add-on with dental features, for example, given a shape and/or orientation of a slider inlet with respect to the add-on distal portion and/or a size and/or position of the add-on body and/or smartphone with respect to the oral opening. For example, where check tissue, in some embodiments, prevents accessing molars using the distal portion of the add-on from certain directions and/or ranges of directions.

A potential disadvantage of changing the orientation of the add-on during scanning of a jaw, for example, as illustrated in FIG. 14B, is that generation of a model from the acquired images involves increased complexity. In some embodiments, when there is a change of orientation, there is overlap in imaged dental regions between images collected prior and after to the change in orientation.

FIG. 14C is a simplified schematic top view of an add-on 1404 with respect to dental features 1464, according to some embodiments.

Where, for example, a slider 1414 of add-on 1404 rotates with respect to add-on body 1404. In some embodiments, rotating mirror/s 320, 322 e.g., so that the mirrors continue to direct light to sides of dental feature/s.

Referring to FIGS. 3A-C, in some embodiments, mirror 324 remains in position with respect to body 1404 and mirrors 320, 322, rotate e.g., with movement of the slider along dental arch 1464.

In some embodiments, mirror 324 rotates with mirrors 320, 322. Where, for example, in some embodiments, element 1684 corresponds to mirror 324.

FIG. 15 is a simplified schematic top view of an add-on 1404 connected to a smartphone 302, with respect to dental features 1464, according to some embodiments.

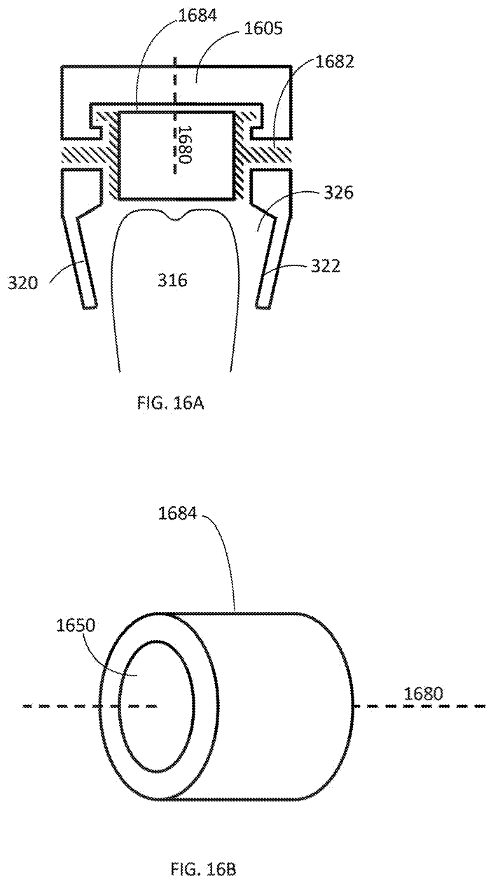

FIG. 16A is a simplified schematic cross section of an add-on, according to some embodiments.

FIG. 16B is a simplified schematic of a portion of an add-on, according to some embodiments.

In some embodiments, mirrors 320, 322 rotate with respect to an add-on body 1605 about axis 1608. Where, in some embodiments, portion 1682 to which the mirrors are attached is able to rotate with respect to add-on body.

In some embodiments, portion 1684 includes a hollow and/or light transmitting channel 1650, potentially enabling light transferred through a distal portion of the add-on to be directed towards mirrors 320, 322.

Where, in some embodiments, FIG. 16B illustrates portion 1650.

FIGS. 14-16B, in some embodiments, relate to embodiments where a head of the scanner add-on e.g., the slider, that is placed on dental feature/s (e.g., onto teeth), is rotatable about an axis.

In some embodiments, a slider is rotatable with respect to a body of an add-on (e.g., slider 1414 and body 1404). Potentially enabling swiping movement along a dental arch, the scanner e.g., from left side to the right side of the mouth and/or allowing a user to perform scanning without having to remove the add-on from the mouth and/or to scan using fewer swipes.

The hollow axis can transfer the light from the projector to the tooth and from the tooth to the camera.

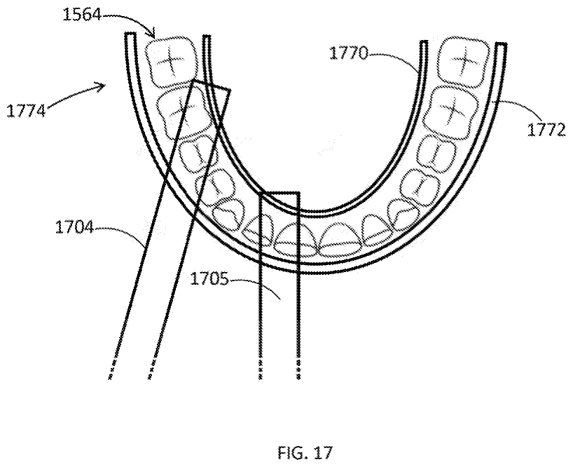

FIG. 17 is a simplified schematic top view of an add-on with respect to dental features, according to some embodiments.

In some embodiments, an element 1774 which remains stationary with respect to dental features 1564 and/or an add-on 1704, 1705 includes mirrors 1770, 1772. Where, in some embodiments, an add-on moving along dental features 1564 e.g., during a swipe motion, e.g., from 1704 to 1705, is optically (and optionally mechanically) coupled to mirrors 1770, 1772 receiving reflections therefrom and transferring the reflections to an imager e.g., of a smartphone attached to the add-on.

In some embodiments, element 1774 has a body (not illustrated which hosts mirrors 1770, 1772) including a shape sized and/or shaped to hold a dental arch or portion thereof. In some embodiments, element 1774 has a gum-guard shape, closed at ends around most distal molars. In some embodiments, element is tailored to an individual.

### Exemplary Scanning of Both Arches

In some embodiments, after the user has finished scanning his full arch and, for example, in order to reduce the accumulated error, the user uses and/or assembles and uses an add-on which projectors and images opposite directions (e.g., 180 degrees apart).

Using this add-on, and placing the add-on and/or smartphone in the middle of the mouth, in some embodiments, allows capture of both back ends of the jaw e.g., in a single frame.

Calculating the depth for each half FOV, in some embodiments, is used to determine the distance of each dental arch end from the camera, e.g., at the same time. This measure, in some embodiments, does not have an accumulated error, associated with capture at the same time, and, in some embodiments, is used to reduce accumulated error of a full jaw scan.

In some embodiments, reducing the error is by adding a constraint to a full arch reconstruction that force the distance between the two edges to be the distance between the two distances of dental arch from the camera that were determined.

In some embodiments, distances between other area/s across the arch determined using distance to the camera (e.g., as described above) are used as constraints in reconstruction.

FIG. 18A is a simplified schematic top view of an add-on 1804 connected to a smartphone 302, with respect to dental features, according to some embodiments.

In some embodiments, add-on 1804 transfers light projected by one or more projector 1808 to dental features 1816, 1817, of both dental arches.

In some embodiments, add-on 1804 has two projectors 1808, or one projector e.g., split using mirror/s.

FIG. 18B is a simplified schematic of an add-on 1804 connected to a smartphone 302, with respect to dental features, according to some embodiments.

In some embodiments, add-on 1804 transfers a FOV of an imager 306 to dental features 1816, 1817, of both dental arches.

In some embodiments, features of FIG. 18A and FIG. 18B are combined into a single add-on.

In some embodiments, image/s of both dental arches are collected simultaneously and/or without removing the add-on from the mouth.

Sharp tips in FIGS. 18A-B, in some embodiments, are rounded externally and/or have a soft covering.

Optionally, in some embodiments, add-on 1804 includes one or more slider (not illustrated in FIGS. 18A-B). Where, in some embodiments, a single slider is contacted to a first dental arch and the second (opposing) dental arch is imaged, in some embodiments with a larger separation between the add-on and the second dental arch than the first dental arch. In some embodiments, the first dental arch is imaged from more than one direction (e.g., FOV splitting using a slider e.g., as described elsewhere in this document). Where, in some embodiments, the second dental arch is imaged from a single direction. In some embodiments, the first dental arch is scanned (e.g., the slider contacting the dental features of the first dental arch) and then the second dental arch is scanned with the slider in contact thereto.

In some embodiments, scanning of the second arch to acquire a coarse model of the jaw and then an additional, 3 FOVs scan, is performed on the second arch e.g., to acquire a more detailed scan. In some embodiments, coarse scan/s are used in stitching images to generate a model.

FIG. 18C is a simplified schematic cross section view of an add-on 1807, according to some embodiments.

In some embodiments, a subject bites onto add-on 1807 upper and lower dental features 1816, 1817 entering into upper 326 and lower cavities 327 of a slider of the add-on. In some embodiments, one or more illuminator 1808, 1809, direct light towards the dental features 1816 e.g., one illuminator illuminating each dental arch. Where, in some embodiments, light is directed to side/s of the dental feature/s 1816, 1817 by mirror/s 320, 321, 322, 323.

### Exemplary Single Optical Element Pattern Projection

FIG. 20A is a simplified schematic of an optical element, according to some embodiments.

FIG. 20B is a simplified schematic of an optical element, according to some embodiments.

In some embodiments, a projector is provided by an optical element/s optically coupled to an illuminator of a smartphone. In some embodiments, a single optical element is coupled, where the optical element includes optical power and patterning. For example, as illustrated in FIG. 20A which illustrates an optical element including a lens and patterning (dashed line) on the surface of the lens. For example, as illustrated in FIG. 20B where patterning (dashed line) is incorporated into a lens.

In some embodiments, the pattern and the projection lens are manufactured as a single optical element, for example using wafer optics to reduce the cost and to allow no assembly product.

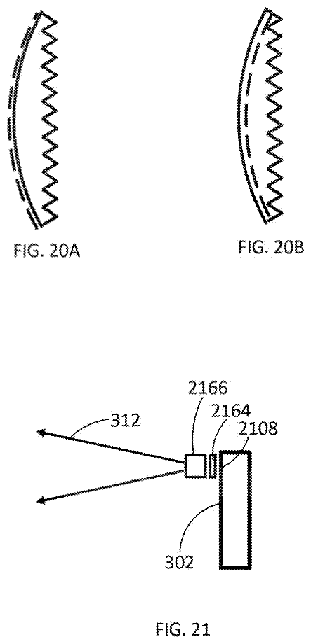

FIG. 21 is a simplified schematic of a projector, according to some embodiments.

In some embodiments, a mobile phone flash 2108 is used for producing patterned light. In some embodiments, light emanating from the smartphone is not directed through the periscope, emanating directly from smartphone 302.

In some embodiments the mobile phone flash 2108 is at least partially covered by a mask 2164 with the pattern to be projected. In some embodiments, the mask pattern is projected over the teeth through a projection lens 2166.

Potentially, projecting directly from smartphone 2108 increases accuracy of scanning of larger portion/s of the mouth e.g., increasing modelling of a full dental arch (and/or at least a quarter, or at least a half arc) using images acquired of the dental features illuminated from patterned light projected directly from smartphone 2108.

In some embodiments, for example, where the internal flash of the smartphone is used for illumination, for example as described regarding FIG. 6 and/or FIG. 21, an add-on does not include electronics (projector LED, LED driver, battery, charging circuit, sync circuit) a potential advantage being potentially reducing cost of the add-on. In some embodiments, smartphone processing is used to synchronize illumination from one or more smartphone illuminator (e.g., smartphone LED and flash) and/or and imager.

In some embodiments, mobile phone flash 2108 is an illumination source for a pattern projector is where patterned light is then transferred through the periscope for example, by an additional at least one mirror, e.g., including one or more feature of light transfer from 608 by add-on 604 of FIG. 6.

In some embodiments, the pattern projector does not include a lens, the pattern directly illuminating (and/or directly being transferred to illuminate) dental feature/s without passing though lens/s of the projector. Potentially, lack of a projector lens reduces cost and/or complexity of the add-on e.g., potentially making a single-use add-on financially feasible.

In some embodiments, elements 2164 and 2166 are provided by a single optical component providing optical power (e.g., to focus the light) and a pattern e.g., the element including one or more feature as illustrated in and/or described regarding FIG. 20A and/or FIG. 20B.

### Exemplary Self-Scanning

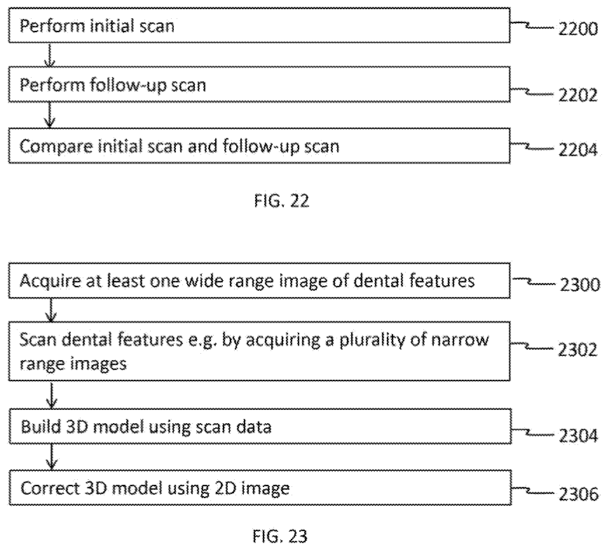

FIG. 22 is a flowchart of a method of dental monitoring, according to some embodiments.

At 2200, in some embodiments, an initial scan is performed.

At 2202, in some embodiments, a follow-up scan is performed.

At 2204, in some embodiments, the initial scan and follow-up scan are compared.

In some embodiments, a subject is monitored using follow-up scan data which, in some embodiments, is acquired by self-scanning. In some embodiments, a detailed initial scan (or more than one initial scan) is used along with follow-up scan data to monitor a subject. In some embodiments, the initial scan being updated using the follow-up scan and/or the follow-up scan being compared to the initial scan to monitor the subject.

In some embodiments, initial scan and/or follow-up scans are performed by:
consumers for initial scan
consumers for Periodic follow ups
GP dentists for initial scan and/o periodic follow ups
Orthodontics for initial scan and/or periodic follow ups
DSO dentists for initial scan and/or periodic follow ups

In some embodiments the user scans his teeth for follow up to a procedure, for example an orthodontic teeth alignment.

In this case, for example differing from a full model of all of the teeth from all sides which is accurate e.g., used to plan a treatment, the follow up scan, in some embodiments, uses prior knowledge, for example the first, accurate model.

In some embodiments, it is assumed that teeth are rigid, and that the full 3D model is accurate and/or of every tooth.

In some embodiments, additional (e.g., follow-up) scans are used to adjust the 3D model e.g., scanning of just a buccal (or lingual) side of teeth, the data from which is registered to the opposing side; lingual (or buccal) of the full model side. In some embodiments, additional (e.g., follow-up) scans are performed when the two arches are closed (e.g., subject biting) and/or are scanned together in a single swipe.

In some embodiments, the periscope is not inserted into the mouth and/or a pattern sticker on the flash is used for scanning the closed bite (e.g., as described elsewhere in this document).

In some embodiments, follow-up scan/s (optionally along with the full scan) are used to track an orthodontic treatment progress, e.g., to send an aligner and/or provide a user with instructions to move to the next aligner that he has. In some embodiments, new aligners are designed during the treatment using follow-up scan data.

In some embodiments, scan/s (optionally along with the full scan) are used to provide information to a dental health practitioner e.g., instead of the user coming to the dentist clinic. Potentially, condition/s (e.g., bleeding and/or cavities) are detected without the presence of the patient in the clinic.

### Exemplary Self-Scanning User Interface (UI)

In some embodiments, (e.g., during self-scanning) the user receives feedback on regarding the scanning.

For example, as described elsewhere in this documents, in some embodiments, a small number (e.g., 1-10, or lower or higher or intermediate numbers or ranges) of swipes are performed e.g., to collect image data from all the teeth inside the mouth from three sides (occlusal, lingual, buccal).

In some embodiments, a coarse 3D model of the patient dental features is built e.g., in real time as the user scans.

In some embodiments, the model is displayed, as it is generated, for example, providing feedback to the person who is scanning e.g., the subject. The display potentially guiding the user as to which region/s use additional scanning.

In some embodiments, potentially more understandable by a lay person, one or more additional or alternative feedback is provided to the user e.g., during and/or after scanning.

In some embodiments the user is guided to scan in a predefined order, for example, by an animation and/or other cues (e.g., aural, haptic). In some embodiments, feedback is provided to the user indicating if the scanning complies with guidance.

For example, in some embodiments one or more progress bar is displayed to the user, where the extent of filling of the bar is according to scan data acquired.

For example, where 100% inculcates scanning of a full mouth e.g., by perform 4 swipes (e.g., a swipe for each half jaw). In some embodiments, at the end of the first swipe 25% of the progress bar is filled, and at the end of all the 4 swipes 100% of the progress bar are filled.

In some embodiments an abstract progress model includes orientation information. For example, in some embodiments, a circle displayed is filled with the proportion of scanning completed. Where, in some embodiments, the portion of the circle filled corresponds to a portion of the mouth.

In some embodiments, the add-on includes in inertial measurement unit (IMU) and/or an IMU of the smartphone is used to provide orientation and/or movement information regarding scanning. In some embodiments, IMU data is used to identify which portion/s of the mouth have been scanned and/or are being scanned.

IMU measurements, in some embodiments, is used to detect if the smartphone and/or the add-on are facing up or down e.g., to determine if the user is currently scanning the upper or lower jaw. IMU measurements, in some embodiments, are used to verify if the user is scanning different side of the mouth e.g., by using a compass to detect the orientation of the smartphone which changes angle when changing scanned mouth side, assuming the head is not moving too much (e.g., by up to 10 or 20 degrees) during the progress. Detecting a mouth side, alternatively or additional, in some embodiments, is using a curve orientation determined from scan images and/or scan position and/or path. For example, a left side scan of the lower jaw, in some embodiments, involves scanner clockwise movement, looking from above.

In some embodiments a detailed position of images acquired is presented, for example, within quarter mouth portion/s (e.g., right side of upper jaw). For an example, using a circular graphic where clock number/s and/or portions are activated (e.g., filled) upon scanning of a corresponding portion of the mouth. In some embodiments, a schematic of a mouth is displayed to the user e.g., an indication being shown when relevant portion/s are scanned. In some embodiments a detailed presentation show surfaces of teeth e.g., lingual, buccal and occlusal area e.g., of each quarter (or sub area in the quarter). Potentially beneficial in the case cases where there is a single periscope with single front mirror (mirror 324 only in FIG. 1) and the user makes a lingual and a buccal swipe. The detection of the buccal or lingual can also be done using the IMU and finding the orientation of the scanner with respect to earth. Assuming the user head is facing forward and not down or up we can detect the scanner tilt and color the correct sub area. In some embodiments the model viewing angle is changed according to detected scanned area to allow the user better view of the scanned area.

In some embodiments, a current position of the add-on is indicated in the UI. For example, where a shape (e.g., circle e.g., dental feature representation) being filled (and/or changing color) as the user self-scans includes an indication of the current add-on scanning position.

In some embodiments, the UI instruct the user regarding scanning for example, where next to perform a “swipe”, for example, a visual and/or oral instruction e.g., a representation of a region to scan as demonstrated with respect to a shape (e.g., circle e.g., dental feature representation), for example blinking in purple of the left upper quarter of the circle to indicate a swipe of the upper left area of the mouth. In some embodiments a UI alerts the user if a different than required and/or instructed scan movement is performed e.g., as determined from image/s acquired and/or from IMU measurements. In some embodiments, feedback as to one speed is provided to the user, e.g., through a user interface, for example regarding speed of scanning, e.g., based on speed determined from image/s acquired and/or IMU data.

### Exemplary Reduction of Accumulated Error

FIG. 23 is a flowchart of a method of dental measurement, according to some embodiments.

At 2300, in some embodiments, at least one wide range image of at least a portion of a dental arc is acquired. The wide range view image including, for example, at least 2-5 teeth, or lower or higher or intermediate numbers or ranges of teeth. In some embodiments, the wide range image is a 2D image, for example acquired using non-patterned light. In some embodiments, the wide range image includes one or more frame of a video. For example, in some embodiments, a user, e.g., as part of a self-scanning procedure, acquires video footage of dental feature/s e.g., by moving a smartphone (e.g., directly) and/or a smartphone coupled to an add-on with respect to dental features e.g., while acquiring video. Where, in some embodiments, video frames acquired from a plurality of directions are used.

In some embodiments, wide range image/s and/or video are acquired using an add-on. Alternatively or additionally, in some embodiments, one or more wide range image is acquired using imager/s of the smartphone directly e.g., using a smartphone rear “selfie” imager and/or a front imager (e.g., acquired from a mirror reflection).

In some embodiments, the wide range (e.g., 2D image) is acquired using the smartphone coupled to an aligner. Where an aligner, in some embodiments, is coupled to the smartphone (e.g., by a connector) and includes one or mechanical feature which assists a user in aligning the smartphone. In some embodiments, the aligner has one or protrusion (e.g., ridge) and/or one or more cavity when the aligner is coupled to the smartphone. In some embodiments, the protrusions are placed between user lips to assist in aligning the smartphone to the user anatomy. In an exemplary embodiment protrusions elongated and orientated in a same general direction, where the direction of elongation is aligned with the lips when used. In some embodiments, a separation between the protrusions is 3-5 cm. In some embodiments, an add-on (e.g., as described elsewhere in this document includes an aligner where, once the add-on is coupled to the smartphone alignment features (e.g., protrusion/s and/or cavities) are positioned for alignment of the smartphone to user anatomy. In some embodiments, the add-on is able to be coupled to the smartphone in more than one way, for example, having an alignment e.g., for capture of wide range images and having a scanning mode e.g., for capture of narrow range images.

At 2302, in some embodiments, dental features are scanned. For example, by moving a distal portion of an add-on with respect to dental features e.g., as described elsewhere within this document. Where, in some embodiments, scanning includes acquiring close range images e.g., where image/s include at most 2-5, or lower or higher or intermediate ranges or numbers of dental features e.g., teeth.

In some embodiments, step 2300 is performed after step 2302. In some embodiments, steps 2300 and 2302 are performed simultaneously or where acquisitions of the steps alternate e.g., at least once. For example, in some embodiments, while moving the add-on within the mouth, and acquiring short range images, larger range images and/or video is acquired. For example, in some embodiments, prior to and/or after moving and/or during along a number of teeth (e.g., 1-5, 1-10 teeth) in a jaw while acquiring short range images long range image/s and/or video is acquired. At 2304, in some embodiments, build 3D model using scan data. For example, in some embodiments, a 3D model is generated using images acquired in step 2302.

At 2306, in some embodiments, 3D model is corrected using wide range image/s and/or video. Alternatively, in some embodiments, the 3D model is generated using data acquired in both steps 2300 and 2302.

In some embodiment, corrections are performed based on one or more assumption, including:

Calibration of the imager. For example, according to one or more detail as illustrated in and/or described regarding one or more of FIGS. 27A-C.

That distances between teeth are accurate (e.g., short range) from closer (or narrow range) scanning acquired images, even though, in some embodiments, there is an accumulated error over many teeth e.g., over a full arch.

In some embodiments, an algorithm to remove accumulated error includes one or more of the following:

Acquire camera intrinsic calibration. E.g., including one or more of one or more of; effective focal length, distortion, and image center.

Segment the teeth in the obtained 3D model.

Segment the teeth in the set of images.

Find the 3D relation (e.g., 6 DOF) between the obtained 3D model of the full arch and the at least one wide range image, such that the projective projection of the obtained 3D model roughly fits the at least one wide range image.

Fine tune the location and rotation (6 DOF) of each tooth or group of teeth in the 3D model, and calculate its 2D projection, e.g., to reduce the difference between projected 2D image of the 3D model and the at least 1 image.

In some embodiments, a merit function of optimization is used to reduce a difference between a projected 2D image of the 3D model and the at least one wide range image and has a high score from maintaining model distances between adjacent teeth.

Where several wide range images have been acquired, more than one wide range image is used (e.g., all) in the optimization. Where, two or more wide range images, in some embodiments are used to generate a 3D model of a full dental arc 2 which is then used in correction of the 3D model built using acquired scan images.

In some embodiments, the method to reduce accumulated error, such as using at least one image, can be used also for verification that the result is accurate. For example, if the residual error of the merit function is not good enough, the app can warn that the accuracy is not good enough and guide the user to scan again and/or take another image. In some embodiments, in case the residual error of the merit function is not good enough for a specific set of teeth or even a single tooth, the app can ask the user to scan again the specific set of teeth or single tooth. In some embodiments, the at least one image can be used for verification only.

In some embodiments, the method of FIG. 23 is used for reduction of accumulated error for measurements collected by an IOS. Where, in some embodiments, the wide range (e.g., 2D image/s) are collected by a smartphone and/or by the IOS.

In some embodiments, the method of FIG. 23 is used combining the 3D and 2D information obtained with the open configurations e.g., described in FIG. 9A and/or FIG. 10.

### Exemplary Optical Tomography

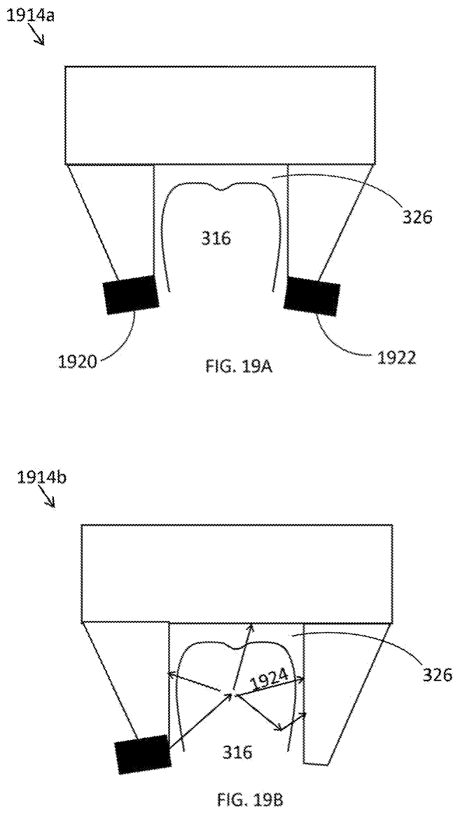

FIG. 19A is a simplified schematic cross sectional view of an add-on, according to some embodiments.

FIG. 19B is a simplified schematic cross sectional view of an add-on, according to some embodiments.

In some embodiments, one or more additional light source 1920, 1922 is attached to an add-on. In some embodiments, at least a proportion of light provided by additional light source/s 1920, 1921 is scattered 1924 by interaction with dental feature/s 316. In some embodiments, the scattered light is gathered through one or more mirror e.g., all 3 mirrors (e.g., mirrors 320, 322, 324 FIG. 3A and FIG. 3B).

In some embodiments, scattered light as gathered by more than one FOV (e.g., emanating from more than one surface of dental features/s) increases information acquired for optical tomography, for example, in comparison to scattered light gathered from fewer directions. In some embodiments, light source/s 1920, 1921 illuminate in one or more of UV, visible, and IR light. In some embodiments, additional illuminator/s 1920/1921 enable transilluminance and/or fluorescence measurements. In some embodiments, the add-on includes at least two light sources 1920, 1922 which are used at different times (e.g., used sequentially and/or alternatively) e.g., potentially increasing the information acquired in images. In some embodiments, illumination from different directions e.g., by illumination incident on different surfaces of a dental feature e.g., as provided illuminators 1920, 1922, on different sides of the dental feature enables determining (e.g., from acquired images) of differential information e.g., relating to properties of differences between the two sides.

In some embodiments, optical tomography e.g., as performed in a self-scan (and/or a plurality of self-scans over time) provides early notice of dental condition onset (e.g., caries) and/or reduces the need for and/or frequency of in-person dental care and/or of x-ray imaging of teeth.

### Exemplary Calibration/s

FIG. 27A is a simplified schematic of an add-on 304 within a packaging 2730, according to some embodiments.

FIG. 27B is a simplified schematic illustrating calibration of a smartphone 302 using a packaging 2830, according to some embodiments.

FIG. 27C is a simplified schematic illustrating calibration of a smartphone 302 attached to an add-on 304 using a packaging, according to some embodiments.

In some embodiments, one or more optical element of smartphone 302 is calibrated, for example, prior to scanning with the smartphone, e.g., scanning with the smart phone 302 attached to the add-on 304.

In some embodiments, packaging 2730 of add-on 304 is used during the calibration.

Where, in some embodiments, packaging 2730 includes a box.

In some embodiments, add-on 304 is provided as part of a kit including the add on and packaging 2730 (and/or an additional or alternative calibration jig). In some embodiments, the kit includes one or more additional calibration element, for example, a calibration target which is moveable and/or positionable e.g., with respect to the packaging and/or to be used without a calibration jig.

Although description in this section is regarding calibration using a box 2730, in some embodiments, box 2730 is an element which is provided separately, and/or is not packaging. In some embodiments, one or more feature of description regarding packaging 2730 are provided by structure/s at a place of purchase of the add-on and/or in a dental office. In some embodiments, the “box” is provided as a printable file.

In some embodiments, box 2730 is used to ship periscope 304 (e.g., as illustrated in FIG. 27A) for example, to the client, is used for calibration. For example, calibration of one or more smartphone cameras, and/or one or more smartphone illuminator (e.g., flash), and/or the periscope 304 alignment e.g., after periscope 304 attachment to the camera and/or flash of the smartphone 302.

In some embodiments, packing box 2730 of the add-on include/s one or more calibration target 2732. Where, in some embodiments, target/s 2732 are located on an inner surface of packaging 2730.

In some embodiments, for example as illustrated in FIG. 27B, calibration is performed by positioning mobile phone 302 with respect to packaging 2730 such that optical element/s of smartphone 302 are aligned with target/s 2732. For example, by placing smartphone 302 on (e.g., as illustrated in FIG. 27B) and/or into packaging 2730. In some embodiments, one or more image (e.g., including target/s 2732) is acquired with smartphone 302 imager/s while aligned with packaging 2730 and/or target/s 2732.

FIG. 27C In some embodiments, packaging 2730 is used for calibration/s and/or validation/s after the add-on is coupled to the smartphone to the smartphone.

In some embodiments, calibration includes imaging one or more target at a known depth from a specific location where the periscope is located. For example, by using a dimension that related to the packaging and/or other element/s housed by and/or provided with the packaging.

In some embodiments, calibration target/s 2732 have known size and/or shape, and/or color (e.g., checkerboard pattern and/or colored squares).

Where, for example, in some embodiments, one or more marking or mechanical guide (e.g., recess, ridge) on packaging 2730 is used to align the add-on and/or smartphone. In some embodiments, a depth of a calibration target from the periscope distal portion is 10 mm or 20 mm or 30 mm, potentially enabling packaging sized to hold the add-on to be used for calibration e.g., where the packaging is (about 20*20*100 mm).

In some embodiments, packaging 2730 includes one or more window 2734 (window/s being either holes in the packaging and/or including transparent material). In some embodiments, window 2734 is located on an opposite side of packaging 2730 body (e.g., an opposite wall to) calibration target 2732.

Where, for example, when smartphone 302 (FIG. 27B) and/or smartphone 302 coupled to add-on 304 (FIG. 27C) for calibration an optical path from the smartphone and/or add-on is through window 2734.

In some embodiments, packaging 2730 includes more than one window and/or more than one calibration target enabling calibration using targets at different distances from the part being calibrated (e.g., smartphone, smartphone coupled to add-on).

In some embodiments, one or more calibration target 2732 is provided by printing onto a surface (e.g., an inner surface e.g., wall) of packaging housing 2730. In some embodiments, one or more calibration target is an element adhered to a surface of packaging 2730 e.g., an inner surface of the packaging.

In some embodiments, a calibration target includes a white colored surface e.g., a white colored surface of packaging 2730. For example, for color calibration e.g., of one or more smartphone illuminator e.g., as described regarding step 208 FIG. 2B.

In some embodiment, packaging 2730 is used more than once, for example, when the add-on is re-coupled to the smartphone (e.g., each time), for example, to verify that coupling is correct.

In some embodiments, calibration target 2732, is used to calibrate colors of light projected by a pattern projector. For example, a color of each line in the pattern as acquired in an image of the patterned light, on the calibration target, after taking into account known color/s of the calibration target on the box are determined. Where, in some embodiments, the colors of the projected light are then verified or adjusted. In some embodiments, a manufacturing process of the packaging is validated to verify the accuracy and/or repeatability of the calibration targets that are produced. In some embodiments, individual packaging is validated after manufacturing.

In some embodiments a calibration target (e.g., as described regarding FIGS. 27A-C and/or regarding step 208 FIG. 2B and/or as describe elsewhere in this document), includes a checkerboard pattern e.g., on at least one inner side wall of the periscope. In some embodiments, where a location of the pattern projector is known relative to the periscope body, it is used for determining location of the periscope and/or the pattern projector e.g., relative to the mobile phone camera e.g., in 6 DOF. The location of the pattern projector relative to the periscope body, in some embodiments, is known e.g., from production according to assembly tolerances. In some embodiments, assembly is by passive and/or active alignment of the location of the pattern projector relative to the periscope body. In some embodiments the location of the pattern projector relative to the periscope body is calibrated in a production line. In some embodiments, information is stored in the cloud and, when the periscope is attached to the mobile phone, (optionally, the periscope is identified) and the calibration information is loaded e.g., from the cloud.

In some embodiments the periscope is designed so that a portion of a light pattern is projected over a periscope inner wall and is within the imager FOV. Where, in some embodiments, this pattern illuminated portion of the inner periscope is used to calibrating a location of the pattern projector relative to the periscope body and/or the camera e.g., in 6 degrees of freedom (DOF). Where the relative location/s are used to correct and/or compensate for movement of the periscope relative to the camera. In some embodiments, a portion of the inner wall is covered with a diffusive reflection layer, such as white coating and/or a white sticker potentially providing increased visibility of the patterned light on the surface.

In some embodiments, calibration includes calibration of positioning of the add-on with respect to the smartphone, for example, positioning of the optical path within the add-on with respect to the smartphone. In some embodiments, calibration is performed and/or re-performed (e.g., enabling frequent compensation) is carried out during imaging e.g., using image/s acquired of the add-on by the imager to be calibrated. For example, inner surfaces of the add-on. Where the image/s include calibration target/s and/or patterned light.

### Exemplary Data Transfer to the Cloud

In some embodiments, for example, where there is a bandwidth limitation between the smartphone and the cloud, the data, in some embodiments, is be transferred in at least two potions including:
Portion 1, which includes a reduced amount of data which is used to generate feedback in real-time regarding the data e.g., feedback to a user. For example, where the data in portion 1 is processed in the cloud and then the feedback is relayed to a user via the smartphone e.g., a user self-scanning and/or to another user e.g., a dental healthcare professional. Where feedback, in some embodiments, is regarding quality and/or completeness and/or clinical analysis and/or.
Portion 2, the full acquired data for generation of a model (e.g., 3D model). Where, the model has sufficient accuracy and/or resolution, for example, for one or more of diagnosis, manufacture of prosthetic/s, manufacture and/or adjustment of aligners.

In some embodiments, e.g., in order to reduce the amount of data that is transferred from a stand-alone camera (e.g., not connected via wire) e.g., is part of the add-on, to the smartphone or from the smartphone to the cloud, data reduction methods are performed on captured images.

In some embodiments, images are cropped is done e.g., to include only the area of the imager acquiring region/s of the mouth, in some embodiments, a final and/or most distal mirror of an add-on (e.g., mirror 324 FIG. 3A). In some embodiments, binning is of a number of pixels, (e.g., where 4 pixels are combined into 1 pixel) is done, in order to reduce the number of pixels per frames.

In some embodiments image/s and/or video acquired is compressed e.g., a lossless compression e.g., a lossy compression. For example, in some embodiments, acquired images are sampled, before sending e.g., to the cloud, for example, were a percentage of frames are sent. For example, 5-25 frames per second or about 15 frames per second, are sampled e.g., where acquisition is at 120 frames per second. Where, in some embodiments, sampling is of 5-20% of acquired frames.

In some embodiments, during real time scanning and transfer of data, stronger data reduction is performed, for example where one or more of, a lower number of FPS are sent, compression is lossy, time binning of data is performed binning. Potentially enabling faster feedback to the user, even in low bandwidth towards to cloud. In some embodiments, the transferred data is used only to provide feedback to a user self-scanning, for example to verify coverage of all required areas of all required teeth during the scan.

In some embodiments, full data is saved locally e.g., on the smartphone and sent to the cloud only after the user has finished self-scanning. The full data, is then, in some embodiments, used to create an accurate model of the user for example, without real time feedback.

In some embodiments, the amount of data reduction for real-time transfer is determined in real-time e.g., using a measure of the upload link bandwidth and/or or the speed of the user scan. Larger bandwidth in the link in some embodiments, is associated with less requirement for reduction of data to be sent and/or slower scan of a specific user potentially allows a lower FPS to be sent.

### Exemplary Add-on

FIG. 24A is a simplified schematic of an add-on 2400 attached to a smartphone 2402, according to some embodiments.

FIG. 24B is a simplified schematic of an add-on 2400, according to some embodiments.

FIG. 24C is a simplified schematic side view of an add-on 2400, according to some embodiments.

In some embodiments, a body 2404 of add-on 2400 extends from a connecting portion 2406 (also herein termed “connector”) of the add-on.

In some embodiments, add-on 2400 includes one or more feature of add-ons as described elsewhere in this document.

In some embodiments, body 2404 extends in a direction which is generally parallel to an orientation of front face 2442 and/or back face 2440 of smartphone 2402. Where, in some embodiments, smartphone front face 2442 hosts a screen of the smartphone and back face 2440 hosts one or more optical element e.g., imager 2420 and/or illuminator.

Where, in some embodiments, the connecting portion 2406 is sized and/or shaped to hold a portion of smartphone 2402. In some embodiments, connecting portion 2404 includes walls 2408 which surround at least partially and/or are adjacent to one or more side 2410 of smartphone 2402. In some embodiments, walls 2408 at least partially surround edges of an end of the smartphone. In some embodiments, walls are connected via a base 2430 of the connector.

In some embodiments, connector 2406 includes an inlet 2412 to an optical path 2414 of add-on 2400. Where, in some embodiments, optical path 2414 transfers light entering the inlet (e.g., from a smartphone optical element e.g., imager 2424) through add-on body 2404.

In some embodiments, optical path 2414 includes one or more mirror 324416, 2418.

In some embodiments, a distal tip of body 2404 includes an angled and/or curved outer surface e.g., surface adjacent to mirror 324416. Where, in some embodiments, an angle of the surface is 10-60 degrees to an angle of outer surfaces of body 2404. The angled surface potentially facilitating positioning of the distal tip into cramped dental position/s e.g., a distal end of a dental arch.

In some embodiments, add-on 2400 includes an illuminator 2422 where, in some embodiments, the illuminator FOV 2424 overlaps that of the smartphone imager 2426. In some embodiments, illuminator 2422 is powered by an add-on power source (not illustrated). Where, in some embodiments, the power source is hosted in the body and/or connector. In some embodiments, illuminator 2422 is powered by the smartphone. In some embodiments, the add-on attached to the smartphone includes an additional illuminator (e.g., of the smartphone e.g., transferred by the add-on and/or of the add-on). In some embodiments, the illuminator illuminates with patterned light and the additional illuminator illuminates with non-patterned light.

### Exemplary Probe

In some embodiments, an add-on includes an elongate element, also herein termed “probe”. Where, in some embodiments, the elongate element is 5-20 mm long, or about 8 mm or about 10 mm or about 12 mm long or lower or higher or intermediate lengths or ranges. In some embodiments the elongate element is 0.1-3 mm wide, or 0.1-1 mm wide or lower or higher or intermediate lengths or ranges. In some embodiments, a length of the elongate element is configured for insertion of the elongate element into the mouth.

In some embodiments, an add-on including a probe does not include a slider.

In some embodiments, the probe is retractable and/or folds away towards a body of the add-on for use of the slider without the probe extending towards dental feature/s. In some embodiments, the probe is unfolded and/or extended so that the probe extends away from a body of the add-on further than the slider, potentially enabling probing of dental features/ e.g., insertion of the probe sub-gingivally, e.g., without the slider contacting gums.

In some embodiments, the user contacts area/s in the mouth with the probe. In some embodiments the user contacts a tooth to measure mobility of the tooth e.g., using one or more force sensor coupled to the probe and/or where mobility is detected from image/s acquired showing the tooth in different location/s with respect to other dental feature/s (e.g., teeth). In some embodiments, a user inputs to a system processor a tooth number and/or location of a tooth to be contacted and/or pushed the processor, in some embodiments adjusting imaging based on the tooth number and/or location.

In some embodiments, at least a portion of the probe is within an FOV of the smartphone imager. In some embodiments, the probe is tracked e.g., position with time. In some embodiments, the probe include a calibration reference, for example a shade reference that can be captured by the smartphone camera and be used to adjust the calibration of the camera in order to get accurate shade measurements. In some embodiments, the camera parameters, for example the focus is changes in order to get a high quality of the calibration reference on the probe.

In some embodiments, the probe includes one or more marker, for example a ball shape on the probe (e.g., of 1 mm diameter). Where, in some embodiments, reflection of light (e.g., patterned) from the ball is tracked in acquired image/s. Tracking the position of the marker allow to detect the position of the probe and it's tip. In some embodiments, knowing the probe position can be used to understand when one tooth end and the other start. An example for doing this is moving the probe over the outer (buccal) side of the teeth and sample the probe tip position. Processing the position will allow the detection of tooth change, for example, using detection of the probe tip position that is more inner (lingual) in areas between the teeth.

In some embodiments, the tip of the probe is thin, for example 200 micron of 100 micron or 400 micron of lower of higher, and are able to enter the interproximal area between two adjacent teeth. When we track the position of the probe tip in the depth images and the 3D model we are able to measure the interproximal distance between two adjacent teeth, for example by touching with the probe tip on the two sides of the gap.

In some embodiments, tracking of the probe while it touches areas inside the mouth is used to calculate force applied by the probe. Calculating the force, in some embodiments, is using advance calibration of the probe e.g., movement of the probe with respect to the applied force. In some embodiments, force measurement/s are used to provide information for dental treatment/s and/or monitoring. For example, touching with the probe on a tooth and measuring its movement while measuring the applied force by the probe, in some embodiments, is used to determine a relationship between force applied to a tooth and its corresponding movement. In some embodiments, this force relationship is used for orthodontic treatment planning, for example to assess tooth root health and/or connection the jawbone and/or suitable forces for correction of tooth location and/or rotation e.g., during an orthodontic treatment.

In some embodiments, the probe is used in transillumination measurements. In some embodiments, light is transmitted into the tooth, for example a lower part of the tooth lingual side and the camera captures transferred light through the tooth emanating from different portion/s of the tooth, e.g., the occlusal and/or buccal part/s of the tooth. In some embodiments, scattered light from the tooth is captured in image/s. In some embodiments, the illumination is at one wavelength and the captured light is at another wavelength e.g., measuring a fluorescence effect by the tooth and/or other material/s e.g., tartar and/or caries. The use of the probe, in some embodiments, enables injection of the light in a particular area (e.g., selected area) e.g., and/or in area/s which are difficult to access e.g., interproximal areas between teeth e.g., near the connection of the tooth and the gum. In some embodiments the light source is located at the probe tip. In some embodiments, the light is transferred to the probe tip using a fiber optic inside a hollow probe. In some embodiments, a light reflecting material is used to cover an inner portion of a hollow probe so that light will reflect from the inner walls until it reaches the probe tip. In some embodiments, the light source is the same light source as the light source for the periscope pattern projector. In some embodiments, a filter for a relevant wavelength range is used. In some embodiments, a different light source will be used, and a synchronization circuit is used e.g., so that the pattern projector and probe tip lighting are not lit at the same time.

In some embodiments, the probe is retracted for use of the add-on without a probe e.g., as described elsewhere in this document. In some embodiments, e.g., after performing a scan and/or during performance of a scan, the probe is extended e.g., to provide other dental measurements e.g., subgingival measurements of dental structure/s. In some embodiments, a user is directed (e.g., by a user interface) when to use the probe e.g., extending (e.g., manually) and/or calibrating the probe. In some embodiments, the probe extends automatically (e.g., via one or more actuator of the add-on) when its use is required.

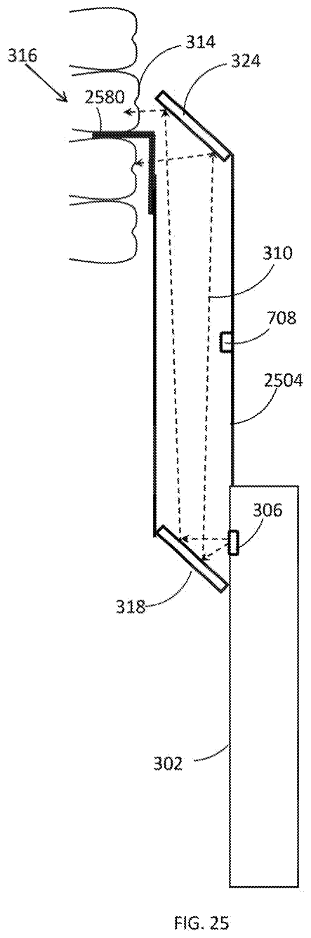

FIG. 25 is a simplified schematic side view of an add-on 2504 connected to a smartphone 302, according to some embodiments.

In some embodiments, add-on 2504 includes an elongated element 2580 (also herein termed “probe”).

In some embodiments, an axis of elongation of elongated element 2580 is non-parallel to a long axis of a body of add-on 2504. Where, in some embodiments, the axis of elongation of elongated element 2580 is 45-90 degrees to the long axis of add-on body.

In some embodiments, elongated element 2580 is sized and/or shaped to be inserted in between teeth, and/or between a dental feature (e.g., tooth) and surrounding gum tissue and/or into a periodontal pocket.

FIG. 26A is a simplified schematic side view of an add-on 2604 connected to a smartphone, according to some embodiments.

FIG. 26B is a simplified cross sectional view of an add-on, according to some embodiments.

In some embodiments, FIG. 26B illustrates a cross sectional view of add-on 2504 of FIG. 26A, e.g., taken across line BB.

In some embodiments, add-on 2604 includes a slider 314 (e.g., as described elsewhere in this document) and one or more elongated element 2580, where, in some embodiments, elongated element/s 2580 are disposed within a cavity 326 of slider 314.

Referring now to FIG. 26B, in some embodiments, add-on 2604 includes one or more than one elongated element. For example, where different elongated elements are sized and/or positioned with respect to add-on 2604 to contact different portions of one or more dental feature e.g., tooth 316 and/or surrounding gums and/or other tissue e.g., cheek and/or tongue.

In some embodiments, elongated element 2580 and/or 2682 include one or more feature as described regarding elongated element 2580 FIG. 25.

FIG. 26C is a simplified cross sectional view of an add-on, according to some embodiments.

FIG. 26D is a simplified cross sectional view of distal end of an add-on, having a probe 2580, where the probe is in a retracted configuration, according to some embodiments.

In some embodiments, a probe 2684a extends perpendicular to a direction of scanning and/or towards a lingual and/or buccal side of dental feature 316 and/or at an angle (e.g., 30-90 degrees, e.g., about perpendicular) to an axis of elongation of add-on body 305 and/or to an axis of extension of slider 314. In some embodiments probe 2684a is inserted into interproximal gaps between teeth e.g., to measure gaps dimensions. In some embodiments probe 2580 includes a light source at its tip which, in some embodiments, is used for detection of cavities and/or other clinical parameters inside the teeth adjacent to the interproximal gap (e.g., using transilluminance and/or other methods described elsewhere in this document) e.g., when inserted into interproximal gap. In some embodiments, probe 2684a is retractable and/or foldable. For example, as illustrated in FIG. 26C by probe 2684b in a folded configuration.

For example, as illustrated in FIG. 26D where probe 2580 (e.g., corresponding to probe 2580 FIG. 26A where in FIG. 26A the probe is extended) is illustrated in a folded configuration.

In some embodiments, probes as described in FIGS. 25, 26A-C are retractable, where, in some embodiments, a portion of the probe extending into space 326 is retractable e.g., into the body of the add-on.

### General

It is expected that during the life of a patent maturing from this application many relevant dental measurement and smartphone technologies will be developed and the scope of the terms dental measurement and smartphone are intended to include all such new technologies a priori.

As used herein the term “about” refers to □20%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of inventions may be presented in a range format. It should be understood that the description in range format is merely for convenience and brevity and should not be construed as an inflexible limitation on the scope of inventions disclosed herein. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of inventions disclosed herein, which are, for clarity, described in the context of separate embodiments, may also be provided in combination in a single embodiment. Conversely, various features of inventions disclosed herein, which are, for brevity, described in the context of a single embodiment, may also be provided separately or in any suitable sub-combination or as suitable in any other described embodiment of inventions disclosed herein. Certain features described in the context of various embodiments are not to be considered essential features of those embodiments, unless the embodiment is inoperative without those elements.

Although inventions have been described in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to inventions disclosed herein. To the extent that section headings are used, they should not be construed as necessarily limiting. In addition, any priority document(s) of this application is/are hereby incorporated herein by reference in its/their entirety.

Inventive embodiments of the present disclosure are also directed to each individual feature, system, apparatus, device, step, code, functionality and/or method described herein. In addition, any combination of two or more such features, systems, apparatuses, devices, steps, code, functionalities, and/or methods, if such features, systems, apparatuses, devices, steps, code, functionalities, and/or methods are not mutually inconsistent, is included within the inventive scope of the present disclosure. Further embodiments may be patentable over prior art by specifically lacking one or more features/functionality/steps (i.e., claims directed to such embodiments may include one or more negative limitations to distinguish such claims from prior art).

The above-described embodiments of the present disclosure can be implemented in any of numerous ways. For example, some embodiments may be implemented (e.g., as noted) using hardware, software or a combination thereof. When any aspect of an embodiment is implemented at least in part in software, the software code can be executed on any suitable processor or collection of processors, servers, and the like, whether provided in a single computer or distributed among multiple computers.

The indefinite articles “a” and “an,” as used herein in the specification and in the claims, unless clearly indicated to the contrary, should be understood to mean “at least one.”

The terms “could”, “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-23. (canceled)

24. A method of dental scanning:
coupling an add-on to a portable electronic device including an imager, said coupling aligning an optical path of said add-on to a FOV of said imager, where said optical path emanates from a distal portion of said add-on which is sized and shaped to be placed within a human mouth;
acquiring, using said imager:
a plurality of narrow range images of one or more dental feature;
at least one wide range image of said one or more dental feature, where said wide range image is acquired from a larger distance from said dental feature than said plurality of narrow range images; and
generating a model of said dental features from said plurality of close range images and said at least one wide range image.

25. The method of dental scanning according to claim 24, wherein said plurality of narrow range images and said at least one wide range image are acquired through said add-on.

26. The method of dental scanning according to claim 24, wherein said acquiring comprises:
acquiring a plurality of narrow range images through said add-on; and
acquiring at least one wide range image by said portable electronic device.

27. The method according to claim 24, wherein said at least one wide range image is acquired through said add-on using an imager FOV which emanates from said add-on distal portion with larger extent than an imager FOV used to acquire said narrow range images.

28. The method according to claim 24, wherein said at least one wide range image is acquired using an imager of said electronic device not coupled to said add-on.

29. The method according to claim 24, wherein said portable electronic device is an electronic communication device having a screen.

30. The method according to claim 24, wherein said model is a 3D model.

31. The method according to claim 24, wherein said generating comprises generating a model using said narrow range images and correcting said model using said at least one wide range image.

32. The method according to claim 24, wherein said plurality of images are acquired of dental features illuminated with patterned light.

33. The method according to claim 24, wherein said add-on optical path transfers patterned light produced by a pattern projector to dental surfaces.

34. The method according to claim 24, wherein said at least one wide range image includes dental features not illuminated by patterned light.

35. A method of dental scanning comprising:
coupling an add-on to a portable electronic device including an imager, said coupling aligning an optical path of said add-on to a FOV of said imager, where said optical path emanates from a distal portion of said add-on which is sized and shaped to be placed within a human mouth;
controlling image data acquired said imager by performing one or more of:
disabling one or more automatic control feature of said electronic device imager; and
determining image processing compensation for said one or more automatic control feature;
acquiring, using said imager, a plurality of images of one or more dental feature;
if imaging processing compensation has been determined, processing said plurality of images according to said processing compensation.

36. The method according to claim 35, wherein said automatic control feature is OIS control.

37. The method according to claim 36, wherein said determining is by using sensor data used by a processor of said electronic device to determine said OIS control.

38. The method according to claim 36, wherein said disabling is by one or more of:
a magnet of said add-on positioned adjacent to said imager; and
software disabling of said OIS control, by software installed on said electronic device.

39-46. (canceled)
